(19) United States
(12) Patent Application Publication
(10) Pub. No.: US 2007/0203134 A1
(43) Pub. Date: Aug. 30, 2007

(54) Benzimidazole Acetonitriles

(75) Inventors: Matthias Schwarz, Geneve (CH); Pascale Gaillard, Collonges Sous Saleve (FR); Patrick Page, Saint-Julien-en-Genenois (FR); Jean-Pierre Gotteland, Beaumont (FR); Russell J. Thomas, Siena (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: APPLIED RESEARCH SYSTEMS ARS HOLDING N.V., Curacao (NL)

(21) Appl. No.: 10/571,470

(22) PCT Filed: Sep. 10, 2004
(86) PCT No.: PCT/EP04/52137

§ 371 Date: Dec. 11, 2006

(51) Int. Cl. A61K 31/5377; A61K 31/506; C07D 413/14; C07D 403/02

(52) U.S. Cl. 514/232.8; 514/275; 544/331; 544/122

(57) ABSTRACT

The present invention is related to benzimidazole acetonitriles as well as to pharmaceutical formulations containing such benzimidazole acetonitriles of formula (I). Said benzimidazole acetonitriles are useful in the treatment of metabolic disorders mediated by insulin resistance or hyperglycemia, comprising diabetes type II, inadequate glucose tolerance, insulin resistance, obesity, polycystic ovary syndrome (PCOS) (I). The present invention is furthermore related to methods of preparing benzoxazole acetonitriles. G is pyrimidinyl; L is an amino group, or a 3-8 membered heterocycloalkyl, containing at least one heteroatom selected from N, O, S or L is an acylamino moiety; R¹ is selected from the group comprising or consisting of hydrogen, sulfonyl, amino, carboxy, aminocarbonyl, C₁-C₆-alkyl, C₂-C₆-alkenyl, C₂-C₆-alkynyl or C₁-C₆-alkoxy, aryl, halogen, cyano or hydroxy; R² is selected from the group comprising or consisting of hydrogen, C₁-C₆-alkyl, C₂-C₆alkenyl, C₂-C₆-alkynyl, or C₁-C₆-alkoxy.

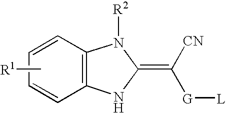

### FIELD OF THE INVENTION

The present invention is related to benzimidazole acetonitriles, as well as pharmaceutical compositions containing such benzimidazole acetonitriles. The compounds of the present invention are useful in the treatment of metabolic disorders mediated by insulin resistance or hyperglycemia, comprising diabetes type II, inadequate glucose tolerance, insulin resistance, obesity, polycystic ovary syndrome (PCOS). In one embodiment, the compounds of the present invention are inhibitors of Glycogen Synthase Kinase 3 (GSK3). The present invention furthermore relates to methods for the preparation of benzimidazole acetonitriles.

### BACKGROUND OF THE INVENTION

Diabetes mellitus is a serious metabolic disease that is defined by the presence of chemically elevated levels of blood glucose (hyperglycemia). The term diabetes mellitus encompasses several different hyperglycemic states. These states include Type 1 (insulin-dependent diabetes mellitus or IDDM) and Type 2 (non-insulin dependent diabetes mellitus or NIDDM) diabetes. The hyperglycemia present in individuals with Type 1 diabetes is associated with deficient, reduced, or nonexistent levels of insulin that are insufficient to maintain blood glucose levels within the physiological range. Conventionally, Type 1 diabetes is treated by administration of replacement doses of insulin, generally by a parenteral route.

Type 2 diabetes is an increasingly prevalent disease of aging. It is initially characterized by decreased sensitivity to insulin and a compensatory elevation in circulating insulin concentrations, the latter of which is required to maintain normal blood glucose levels. As described below, GSK3 inhibition stimulates insulin dependent processes and is consequently viewed to be useful in the treatment of type 2 diabetes. Recent data obtained using lithium salts provides evidence for this notion.

The prevalence of insulin resistance in glucose intolerant subjects is well known. Reaven et al (American Journal of Medicine, 60, 80 (1976)) used a continuous infusion of glucose and insulin (insulin/glucose clamp technique) and oral glucose tolerance tests to demonstrate that insulin resistance exists in a diverse group of non-obese, non-ketotic subjects. These subjects ranged from borderline glucose tolerant to overt, fasting hyperglycemia. The diabetic groups in these studies included both insulin dependent (IDDM) and non-insulin dependent (NIDDM) subjects.

Coincident with sustained insulin resistance is the more easily determined hyperinsulinemia, which may be measured by accurate determination of circulating plasma insulin concentration in the plasma of subjects. Hyperinsulinemia may be present as a result of insulin resistance, such as is in obese and/or diabetic (NIDDM) subjects and/or glucose intolerant subjects, or in IDDM subjects, as a consequence of over injection of insulin compared with normal physiological release of the hormone by the endocrine pancreas.

The association of hyperinsulinemia and insulin resistance with obesity has been well established by numerous experimental, clinical and epidemiological studies (Stout, Metabolism, 34, 7 (1985)).

The association of hyperinsulinemia and insulin resistance with Polycystic Ovary Syndrome (PCOS) is also well acknowledged (Diamanti-Kandarakis et al.; Therapeutic effects of metformin on insulin resistance and hyperandrogenism in polycystic ovary syndrome; European Journal of Endocrinology 138, 269-274 (1998), Andrea Dunaif, Insulin Resistance and the Polycystic Ovary Syndrome: Mechanism and Implications for Pathogenesis; Endocrine Reviews 18(6), 774-800 (1997)).

Type II diabetes mellitus is currently treated with sulfonylureas, biguanides, such as Metformin and thiazolidenediones, such as Troglitazone, Rosiglitazone or Pioglitazone, as oral hypoglycemic agents.

Glycogen synthase kinase 3 (GSK3) is a serine/threonine kinase for which two isoforms, α and β, have been identified (Trends Biochem. Sci., 16 p. 177-81 (1991) by Woodgett et al.). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such glucose transport. Subsequently, it has been shown that GSK3 activity is also inactivated by other growth factors that, like insulin, signal through receptor tyrosine kinases (RTKs). Examples of such signalling molecules include IGF-1 and EGF. GSK3 beta activity is regulated by serine (inhibitory) and tyrosine (stimulatory) phosphorylation, by protein complex formation, and by its intracellular localization. GSK3 beta phosphorylates and thereby regulates the functions of many metabolic, signalling and structural proteins. Notable among the signalling proteins regulated by GSK3 beta are the many transcription factors, including activator protein-1 cells, Myc, beta-catenin, CCAAT/enhancer binding protein, and NFkappaB.

Agents that inhibit GSK3 activity are viewed to be useful in the treatment of type II diabetes.

In the patent literature, different classes of GSK3 inhibitors have been disclosed (e.g. WO 02/20495, Chiron Corporation; WO 02/10141, Pfizer Products Inc.; WO 02/22608, Vertex Pharmaceuticals Inc.).

WO 01/47920 discloses benzazoles of formula (A) in particular for the treatment of neuronal disorders, autoimmune diseases, cancer and cardiovascular diseases.

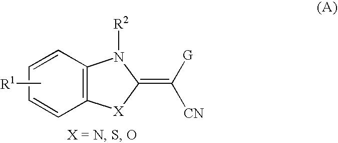

It was now found that certain compounds of formula (A), surprisingly, are in addition useful in the treatment of metabolic disorders mediated by insulin resistance or hyperglycemia, comprising diabetes type II, inadequate glucose tolerance, insulin resistance, obesity, polycystic ovary syndrome (PCOS).

### SUMMARY OF THE INVENTION

The present invention relates to benzimidazole acetonitriles of formula (I) 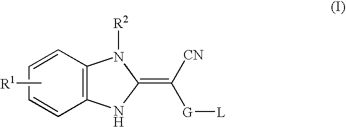
as well as their pharmaceutically acceptable salts.

Also, the present invention relates to the use of compounds of formula (I) as medicament, in particular for the treatment and/or prevention of metabolic disorders mediated by insulin resistance or hyperglycemia, such as diabetes type II, inadequate glucose tolerance, insulin resistance, obesity, polycystic ovary syndrome (PCOS).

### DETAILED DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“C₁-C₆-alkyl” refers to alkyl groups having 1 to 6 carbon atoms. This term is exemplified by groups such as methyl, ethyl, n-propyl, isopropyl, n-butyl, isobutyl, tert-butyl, n-butyl, n-pentyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g. norbornyl). Preferred cycloalkyl include cyclopentyl, cyclohexyl, norbornyl and the like.

“C₁-C₆-alkyl cycloalkyl” refers to C₁-C₆-alkyl groups having a cycloalkyl substituent, including cyclohexylmethyl, cyclopentylpropyl, and the like.

“heterocycloalkyl” refers to a C₃-C₈-cycloalkyl group according to the definition above, in which 1 to 3 carbon atoms are replaced by hetero atoms chosen from the group consisting of O, S, NR, R being defined as hydrogen or C₁-C₆ alkyl. Preferred heterocycloalkyl include pyrrolidine, piperidine, piperazine, 1-methylpiperazine, morpholine, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having a carboxy substituent, including 2-carboxyethyl and the like.

“Acyl” refers to the group —C(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkynyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxycarbonyl” refers to C₁-C₆-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl amino” refers to C₁-C₆-alkyl groups having an amino substituent, including 2-(1-pyrrolidinyl)ethyl and the like.

“Ammonium” refers to a positively charged group —N⁺RR′R″, where each R, R′,R″ is independently, “C₁-C₆-alkyl”, “C₂-C₆-alkenyl”, “C₂-C₆-alkynyl”, “C₃-C₈-cycloalkyl”, “heterocycloalkyl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₆-alkyl groups having a sulfonyloxy substituent, including 2-(methylsulfonyloxy)ethyl and the like.

“Sulfonyl” refers to group “—SO₂—R“ wherein R is selected from H, “aryl”, “heteroaryl”, “C₁-C₆-alkyl”, “C₁-C₆-alkyl” substituted with halogens, e.g., an —SO₂-CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₆-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₆-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl”or “C₁-C₆-alkyl heteroaryl”,

“C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “carbamate”, “aryl”, “heteroaryl”, “sulfinyl”, “sulfonyl, “alkoxy”, “sulfanyl”, “halogen”, “carboxy”, trihalomethyl, cyano, hydroxy, mercapto, nitro, and the like. Alternatively, said substitution could also comprise situations where neighbouring substituents have undergone ring closure, notably when vicinal functional substituents are involved, thus forming, e.g., lactams, lactons, cyclic anhydrides, but also acetals, thioacetals, aminals formed by ring closure for instance in an effort to obtain a protective group.

“Pharmaceutically acceptable salts or complexes” refers to salts or complexes of the below-identified compounds of formula (1) that retain the desired biological activity. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, methanesulfonic acid and poly-galacturonic acid. Said compounds can also be administered as pharmaceutically acceptable quaternary salts known by a person skilled in the art, which specifically include the quarternary ammonium salt of the formula —NR,R′,R″⁺Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkly, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

A first aspect of the invention consists in benzimidazole acetonitriles of formula I: 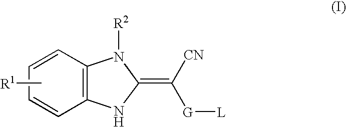
G is an unsubstituted or substituted pyrimidinyl.

In particular, G may be either of the substituted pyrimidinyl moieties 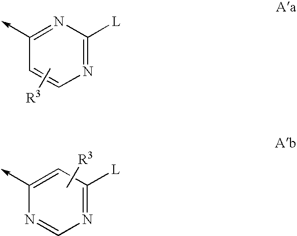
L is an amino group, or an unsubstituted or a substituted 3-8 membered heterocycloalkyl, containing at least one heteroatom selected from N, O, S or L is an acylamino moiety.

R¹ is selected from the group comprising or consisting of hydrogen, sulfonyl, amino, carboxy, amino carbonyl, unsubstituted or substituted C₁-C₆-alkyl, unsubstituted or substituted C₂-C₆-alkenyl, unsubstituted or substituted C₂-C₆-alkynyl or C₁-C₆-alkoxy, unsubstituted or substituted aryl (e.g. phenyl), halogen, cyano or hydroxy.

Preferably R¹ is H or C₁-C₃ alkyl (e.g. a methyl or ethyl group).

R² is selected from the group comprising or consisting of hydrogen, unsubstituted or substituted C₁-C₆-alkyl, unsubstituted or substituted aryl (e.g. phenyl), unsubstituted or substituted C₂-C₆-alkenyl, unsubstituted or substituted C₂-C₆-alkynyl, unsubstituted or substituted cycloalkyl or C₁-C₆-alkoxy.

Preferably R² is a C₁-C₃ alkyl (e.g. an ethyl group).

R³ is selected from the group comprising or consisting of hydrogen, unsubstituted or substituted C₁-C₆-alkyl, unsubstituted or substituted aryl (e.g. phenyl), unsubstituted or substituted C₂-C₆-alkenyl, unsubstituted or substituted C₂-C₆-alkynyl, unsubstituted or substituted cycloalkyl or C₁-C₆-alkoxy.

Preferably R³ is hydrogen or a C₁-C₃ alkyl (e.g. a methyl or an ethyl group).

The compounds of the present invention also comprises their tautomers, their geometrical isomers, their optically active forms as enantiomers, diastereomers and its racemate forms, as well as pharmaceutically acceptable salts thereof. Preferred pharmaceutically acceptable salts of the formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, trifluoroacetate, and para-toluenesulfonate salts.

More specifically, the benzimidazole acetonitriles of the present invention comprise the tautomeric forms, e.g. the below ones:

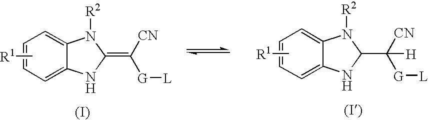

A specific embodiment of the present invention consists in benzimidazole acetonitriles of formula (Ia) in its tautomeric forms, e.g. the below ones: 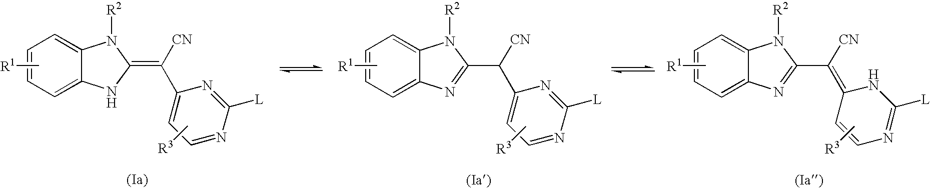
R¹, R², R³ and L are as defined for formula (I).

According to a specific embodiment, the moiety L is an amino group of the formula —NR⁵R⁶ wherein R⁵ and R⁶ are each independently from each other H, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), unsubstituted or substituted C₁-C₆-alkyl aryl, unsubstituted or substituted C₁-C₆-alkyl heteroaryl, unsubstituted or substituted C₁-C₆-alkenyl aryl unsubstituted or substituted C₁-C₆-alkenyl heteroaryl, unsubstituted or substituted C₁-C₆-alkynyl aryl unsubstituted or substituted C₁-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₁-C₆-alkenyl cycloalkyl, unsubstituted or substituted C₁-C₆-alkenyl heterocycloalkyl, unsubstituted or substituted C₁-C₆-alkynyl cycloalkyl, unsubstituted or substituted C₁-C₆-alkynyl heterocycloalkyl.

Alternatively, R⁵ and R⁶ may form a ring together with the nitrogen to which they are bound.

In a specific embodiment, R⁵ is hydrogen or a methyl or ethyl or propyl group and R⁶ is selected from the group consisting of unsubstituted or substituted C₁-C₆-alkyl, unsubstituted or substituted C₁-C₆ alkyl-aryl, unsubstituted or substituted C₁-C₆-alkyl-heteroaryl, unsubstituted or substituted cycloalkyl, unsubstituted or substituted heterocycloalkyl, unsubstituted or substituted aryl or heteroaryl and unsubstituted or substituted 4-8 membered saturated or unsaturated cycloalkyl.

In a preferred embodiment R⁵ is H and R⁶ is selected from the group consisting of C₁-C₆ alkyl, 3-8 membered cycloalkyl, 3-8 membered heterocycloalkyl, heteroaryl, C₁-C₆-alkyl heteroaryl, C₁-C₆-alkyl cycloalkyl, C₁-C₆-alkyl heterocycloalkyl. Examples of cycloalkyl are cyclopropyl, cyclopentyl or cyclohexyl.

More specifically R⁶ may be a C₂-C₄ alkyl, in particular an ethylene or propylene moiety, optionally substituted with an unsubstituted or substituted heteroaryl group, e.g., an unsubstituted or substituted pyridyl or a 2-pyrrolidinone (2-oxopyrrolidine) or a triazolyl moiety.

According to a further specific embodiment, the moiety L is an acylamino moiety of the formula —NR⁵C(O)R⁶ wherein R⁵ and R⁶ are each independently from each other H, unsubstituted or substituted C₁-C₆-alkyl, unsubstituted or substituted C₂-C₆-alkenyl unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, unsubstituted or substituted C₁-C₆-alkyl aryl, unsubstituted or substituted C₁-C₆-alkyl heteroaryl, unsubstituted or substituted C₁-C₆-alkenyl aryl, unsubstituted or substituted C₁-C₆-alkenyl heteroaryl, unsubstituted or substituted C₁-C₆-alkynyl aryl, unsubstituted or substituted C₁-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₁-C₆-alkenyl cycloalkyl, unsubstituted or substituted C₁-C₆-alkenyl heterocycloalkyl, unsubstituted or substituted C₁-C₆-alkynyl cycloalkyl, unsubstituted or substituted C₁-C₆-alkynyl heterocycloalkyl.

Specific benzimidazole acetonitriles according to formula (I) include:
(2Z)-(1-ethyl-1,3-dihydro-2H-benzimidazol-2-ylidene)(2-{[3-(1H-pyrazol-1-yl)propyl]-amino}pyrimidin-4-yl)acetonitrile
(2Z)-(1-ethyl-1,3-dihydro-2H-benzimidazol-2-ylidene)(5-methyl-2-{[3-(1H-pyrazol-1-yl)propyl]amino}pyrimidin-4-yl)acetonitrile
(2Z)-[2-(cyclobutylamino)-5-methylpyrimidin-4-yl](1-ethyl-1,3-dihydro-2H-benzimidazol-2-ylidene)acetonitrile
(2Z)-(1-ethyl-1,3-dihydro-2H-benzimidazol-2-ylidene)(5-methyl-2-{[3-(2-oxopyrrolidin-1-yl)propyl]amino}pyrimidin-4-yl)acetonitrile
(2Z)-(1-ethyl-1,3-dihydro-2H-benzimidazol-2-ylidene)(2-{[3-(2-oxopyrrolidin-1-yl)propyl]amino}pyrimidin-4-yl)acetonitrile
(2Z)-(1-ethyl-1,3-dihydro-2H-benzimidazol-2-ylidene)(2-{[3-(1H-1,2,4-triazol-1-yl)propyl]amino}pyrimidin-4-yl)acetonitrile
(2Z)-(1-ethyl-1,3-dihydro-2H-benzimidazol-2-ylidene)(5-methyl-2-{[3-(1H-1,2,4-triazol-1-yl)propyl]amino}pyrimidin-4-yl)acetonitrile
[2-(cyclopentylamino)-5-methylpyrimidin-4-yl](1-ethyl-1H-benzimidazol-2-yl)acetonitrile
(2Z)-(2-{[3-(2-oxopyrrolidin-1-yl)propyl]amino}pyrimidin-4-yl)(1-propyl-1,3-dihydro-2H-benzimidazol-2-ylidene)acetonitrile
(1-ethyl-1H-benzimidazol-2-yl){5-methyl-2-[(2-pyridin-3-ylethyl)amino]pyrimidin-4-yl}acetonitrile
(2Z)-[2-(cyclobutylamino)pyrimidin-4-yl](1-ethyl-1,3-dihydro-2H-benzimidazol-2-ylidene)acetonitrile
(2Z)-[2-(cycloheptylamino)-5-methylpyrimidin-4-yl](1-ethyl-1,3-dihydro-2H-benzimidazol-2-ylidene)acetonitrile
[2-(cyclopentylamino)pyrimidin-4-yl](1-ethyl-1H-benzimidazol-2-yl)acetonitrile
1,3-dihydro-2H-benzimidazol-2-ylidene(5-methyl-2-{[3-(2-oxopyrrolidin-1-yl)propyl]amino)pyrimidin-4-yl)acetonitrile
(2Z)-(1-cyclobutyl-1,3-dihydro-2H-benzimidazol-2-ylidene)(2-{[3-(2-oxopyrrolidin-1-yl)propyl]amino}pyrimidin-4-yl)acetonitrile
(1-ethyl-1H-benzimidazol-2-yl){2-[(2-pyridin-3-ylethyl)amino]pyrimidin-4-yl}acetonitrile
(2Z)-(1-ethyl-1,3-dihydro-2H-benzimidazol-2-ylidene)[2-(isobutylamino)-5-methylpyrimidin-4-yl]acetonitrile
(2Z)-(1-ethyl-1,3-dihydro-2H-benzimidazol-2-ylidene)(2-{[2-(1H-imidazol-4-yl)ethyl]amino}pyrimidin-4-yl)acetonitrile
(2Z)-(1-ethyl-1,3-dihydro-2H-benzimidazol-2-ylidene)[2-(isobutylamino)pyrimidin-4-yl]acetonitrile
[2-(cyclopropylamino)pyrimidin-4-yl](1-ethyl-1H-benzimidazol-2-yl)acetonitrile
[2-({2-[6-(dimethylamino)pyridin-3-yl]ethyl}amino)pyrimidin-4-yl](1-ethyl-1H-benzimidazol-2-yl)acetonitrile
(1-ethyl-1H-benzimidazol-2-yl)(2-{[2-(1H-1,2,4-triazol-1-yl)ethyl]amino}pyrimidin-4-yl)acetonitrile
(2Z)-(1-ethyl-1,3-dihydro-2H-benzimidazol-2-ylidene)(2-{[2-(1H-imidazol-4-yl)ethyl]amino}-5-methylpyrimidin-4-yl)acetonitrile
[2-({2-[6-(dimethylamino)pyridin-3-yl]ethyl}amino)-5-methylpyrimidin-4-yl](1-ethyl-1H-benzimidazol-2-yl)acetonitrile
(2Z)-[2-(cycloheptylamino)pyrimidin-4-yl](1-ethyl-1,3-dihydro-2H-benzimidazol-2-ylidene)acetonitrile
[2-(cyclopropylamino)-5-methylpyrimidin-4-yl](1-ethyl-1H-benzimidazol-2-yl)acetonitrile
(1-ethyl-1H-benzimidazol-2-yl){2-[(2-pyridin-2-ylethyl)amino]pyrimidin-4-yl}acetonitrile
[2-(cyclopentylamino)-5-methylpyrimidin-4-yl](1,3-dihydro-2H-benzimidazol-2-ylidene)acetonitrile
[2-(cyclohexylamino)pyrimidin-4-yl](1-ethyl-1H-benzimidazol-2-yl)acetonitrile
(2Z)-(1-ethyl-1,3-dihydro-2H-benzimidazol-2-ylidene)(2-{[2-(1H-indol-3-yl)ethyl]amino}-5-methylpyrimidin-4-yl)acetonitrile
(1-ethyl-1H-benzimidazol-2-yl){5-methyl-2-[(2-pyridin-2-ylethyl)amino]pyrimidin-4-yl}acetonitrile
{2-[(2-ethoxyethyl)amino]pyrimidin-4yl}(1-ethyl-1H-benzimidazol-2-yl)acetonitrile
(1-ethyl-1H-benzimidazol-2-yl){5-methyl-2-[(1-methylbutyl)amino]pyrimidin-4-yl}acetonitrile
(1-ethyl-1H-benzimidazol-2-yl)[2-(methylamino)pyrimidin-4-yl]acetonitrile
(1-ethyl-1H-benzimidazol-2-yl)(5-methyl-2-{[2-(1H-pyrazol-1-yl)ethyl]amino}pyrimidin-4-yl)acetonitrile
1H-benzimidazol-2-yl{5-methyl-2-[(2-pyridin-3-ylethyl)amino]pyrimidin-4yl}acetonitrile
(2Z)-(1-ethyl-1,3-dihydro-2H-benzimidazol-2-ylidene)(2-{[2-(1H-imidazol-1-yl)ethyl]amino}-5-methylpyrimidin-4-yl)acetonitrile
1H-benzimidazol-2-yl{2-[(2-pyridin-3-ylethyl)amino]pyrimidin-4-yl}acetonitrile
(1-ethyl-1H-benzimidazol-2-yl){2-[(1-methylbutyl)amino]pyrimidin-4-yl acetonitrile
{2-[(cyclohexylmethyl)amino]-5-methylpyrimidin-4-yl}(1-ethyl-1H-benzimidazol-2-yl)acetonitrile
1H-benzimidazol-2-yl[2-(cyclopentylamino)pyrimidin-4-yl]acetonitrile
(1-ethyl-1H-benzimidazol-2-yl){6-methyl-2-[(2-pyridin-3-ylethyl)amino]pyrimidin-4-yl}acetonitrile
1H-benzimidazol-2-yl[2-(cyclopropylamino)pyrimidin-4-yl]acetonitrile
[2-(cyclopentylamino)-6-methylpyrimidin-4-yl](1-ethyl-1H-benzimidazol-2-yl)acetonitrile
{2-[(cyclohexylmethyl)amino]pyrimidin-4-yl}(1-ethyl-1H-benzimidazol-2-yl)acetonitrile
(1-ethyl-1H-benzimidazol-2-yl){6-[(2-pyridin-3-ylethyl)amino]pyrimidin-4-yl}acetonitrile.
(1-ethyl-1H-benzimidazol-2-yl){2-[(3-pyrrolidin-1-ylpropyl)amino]pyrimidin-4-yl}acetonitrile
(1-ethyl-1H-benzimidazol-2-yl)[2-(4-ethylpiperazin-1-yl)-5-methylpyrimidin-4-yl]acetonitrile
(1-ethyl-1H-benzimidazol-2-yl){2-[(2-furylmethyl)amino]-5-methylpyrimidin-4-yl}acetonitrile
(2Z)-(1-ethyl-1,3-dihydro-2H-benzimidazol-2-ylidene){5-methyl-2-[(1-methylpiperidin-4-yl)amino]pyrimidin-4-yl}acetonitrile
(2Z)-[2-(cyclohexylamino)-5-methylpyrimidin-4-yl](1-ethyl-1,3-dihydro-2H-benzimidazol-2-ylidene)acetonitrile
(2Z)-[2-(ethylamino)-5-methylpyrimidin-4-yl](1-ethyl-1,3-dihydro-2H-benzimidazol-2-ylidene)acetonitrile
[2-(cyclopentylamino)-5-methylpyrimidin-4-yl](1,3-diethyl-1,3-dihydro-2H-benzimidazol-2-ylidene)acetonitrile
(2Z)-(1-ethyl-1,3-dihydro-2H-benzimidazol-2-ylidene)[5-methyl-2-(piperidin-4-ylamino)pyrimidin-4-yl]acetonitrile, and
(2Z)-(1-ethyl-1,3-dihydro-2H-benzimidazol-2-ylidene){5-methyl-2-[(2-piperidin-1-ylethyl)amino]pyrimidin-4-yl}acetonitrile, or it a tautomer form thereof.
N-{4-[(Z)-cyano(1-ethyl-1,3-dihydro-2H-benzimidazol-2-ylidene)methyl]-5-methylpyrimidin-2-yl}-4-(4-methylpiperazin-1-yl)-4-oxobutanamide
N-{4-[(Z)-cyano(1-ethyl-1,3-dihydro-2H-benzimidazol-2-ylidene)methyl]pyrimidin-2-yl}-4-(4-methylpiperazin-1-yl)-4-oxobutanamide
(1R,5R,7R)-N-{4-[(Z)-cyano(1-ethyl-1,3-dihydro-2H-benzimidazol-2-ylidene)methyl]-5-methylpyrimidin-2-yl}-6,8-dioxa-3-azabicyclo[3.2.1]octane-7-carboxamide
(1S,5S,7S)-N-{4-[(Z)-cyano(1-ethyl-1,3-dihydro-2H-benzimidazol-2-ylidene)methyl]-5-methylpyrimidin-2-yl}-6,8-dioxa-3-azabicyclo[3.2.1]octane-7-carboxamide
(1S,4S,5S,7R)-N-{4-[(Z)-cyano(1-ethyl-1,3-dihydro-2H-benzimidazol-2-ylidene)methyl]-5-methylpyrimidin-2-yl}-4-methyl-6,8-dioxa-3-azabicyclo[3.2.1]octane-7-carboxamide
(1S,4R,5S,7R)-N-{4-[(Z)-cyano(1-ethyl-1,3-dihydro-2H-benzimidazol-2-ylidene)methyl]-5-methylpyrimidin-2-yl}-4-methyl-6,8-dioxa-3-azabicyclo[3.2.1]octane-7-carboxamide
N-{4-[(Z)-cyano(1-ethyl-1,3-dihydro-2H-benzimidazol-2-ylidene)methyl]-5-methylpyrimidin-2-yl}piperazine-2-carboxamide
(4S)-N-{4-[(Z)-cyano(1-ethyl-1,3-dihydro-2H-benzimidazol-2-ylidene)methyl]-5-methylpyrimidin-2-yl}-1,3-thiazolidine-4-carboxamide
(4R)-N-{4-[(Z)-cyano(1-ethyl-1,3-dihydro-2H-benzimidazol-2-ylidene)methyl]-5-methylpyrimidin-2-yl}-1,3-thiazolidine-4-carboxamide
N-{4-[(Z)-cyano(1-ethyl-1,3-dihydro-2H-benzimidazol-2-ylidene)methyl]-5-methylpyrimidin-2-yl}-5-oxo-L-prolinamide
4-tert-butyl 1-(9H-fluoren-9-ylmethyl) 2-[({4-[(Z)-cyano(1-ethyl-1,3-dihydro-2H-benzimidazol-2-ylidene)methyl]-5-methylpyrimidin-2-yl}amino)carbonyl]piperazine-1,4-dicarboxylate
tert-butyl (4S)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate
tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-5-oxopyrrolidine-1-carboxylate
tert-butyl (4R)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate
2-(1-acetylpiperidin-4-yl)-N-{4-[(Z)-cyano(1-ethyl-1,3-dihydro-2H-benzimidazol-2-ylidene)methyl]-5-methylpyrimidin-2-yl}acetamide
N-{4-[(Z)-cyano(1-ethyl-1,3-dihydro-2H-benzimidazol-2-ylidene)methyl]-5-methylpyrimidin-2-yl}-4-methylmorpholine-2-carboxamide
4-acetyl-N-{4-[(Z)-cyano(1-ethyl-1,3-dihydro-2H-benzimidazol-2-ylidene)methyl]-5-methylpyrimidin-2-yl}morpholine-2-carboxamide
N-{4-[(Z)-cyano(1-ethyl-1,3-dihydro-2H-benzimidazol-2-ylidene)methyl]-5-methylpyrimidin-2-yl}-4-[(4-methylpiperazin-1-yl)methyl]benzamide
tert-butyl 3-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate
tert-butyl 4-[2-({4-[(Z)-cyano(1-ethyl-1,3-dihydro-2H-benzimidazol-2-ylidene)methyl]-5-methylpyrimidin-2-yl}amino)-2-oxoethyl]piperidine-1-carboxylate
N-{4-[(Z)-cyano(1-ethyl-1,3-dihydro-2H-benzimidazol-2-ylidene)methyl]-5-methylpyrimidin-2-yl}morpholine-2-carboxamide
N-{4-[(Z)-cyano(1-ethyl-1,3-dihydro-2H-benzimidazol-2-ylidene)methyl]-5-methylpyrimidin-2-yl}-2-piperidin-4-ylacetamide
N-{4-[(Z)-cyano(1-ethyl-1,3-dihydro-2H-benzimidazol-2-ylidene)methyl]-5-methylpyrimidin-2-yl}piperidine-3-carboxamide
tert-butyl 2-[({4-[(Z)-cyano(1-ethyl-1,3-dihydro-2H-benzimidazol-2-ylidene)methyl]-5-methylpyrimidin-2-yl}amino)carbonyl]morpholine-4-carboxylate
N-[3-({4-[(Z)-cyano(1-ethyl-1,3-dihydro-2H-benzimidazol-2-ylidene)methyl]-5-methylpyrimidin-2-yl}amino)-3-oxopropyl]benzamide
tert-butyl [2-({4-[(Z)-cyano(1-ethyl-1,3-dihydro-2H-benzimidazol-2-ylidene)methyl]-5-methylpyrimidin-2-yl}amino)-2-oxoethyl]methylcarbamate
N-{4-[(Z)-cyano(1-ethyl-1,3-dihydro-2H-benzimidazol-2-ylidene)methyl]-5-methylpyrimidin-2-yl}-1-methylpiperidine-3-carboxamide
N˜1˜-{4-[(Z)-cyano(1-ethyl-1,3-dihydro-2H-benzimidazol-2-ylidene)methyl]-5-methylpyrimidin-2-yl}-N˜2˜-methylglycinamide
N-{4-[(Z)-cyano(1-ethyl-1,3-dihydro-2H-benzimidazol-2-ylidene)methyl]-5-methylpyrimidin-2-yl}pyrrolidine-3-carboxamide
tert-butyl 3-[({4-[(Z)-cyano(1-ethyl-1,3-dihydro-2H-benzimidazol-2-ylidene)methyl]-5-methylpyrimidin-2-yl}amino)carbonyl]pyrrolidine-1-carboxylate
(2S)-N-{4-[(Z)-cyano(1-ethyl-1,3-dihydro-2H-benzimidazol-2-ylidene)methyl]-5-methylpyrimidin-2-yl}-2,5-dihydro-1H-pyrrole-2-carboxamide
tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-2,5-dihydro-1H-pyrrole-1-carboxylate
N-{4-[(Z)-cyano(1-ethyl-1,3-dihydro-2H-benzimidazol-2-ylidene)methyl]-5-methylpyrimidin-2-yl}-2-(2-methoxyphenyl)acetamide
1-acetyl-N-{4-[(Z)-cyano(1-ethyl-1,3-dihydro-2H-benzimidazol-2-ylidene)methyl]-5-methylpyrimidin-2-yl}piperidine-4-carboxamide
N-{4-[(Z)-cyano(1-ethyl-1,3-dihydro-2H-benzimidazol-2-ylidene)methyl]-5-methylpyrimidin-2-yl}-1-methylpiperidine-4-carboxamide
4-amino-N-{4-[(Z)-cyano(1-ethyl-1,3-dihydro-2H-benzimidazol-2-ylidene)methyl]-5-methylpyrimidin-2-yl}butanamide
tert-butyl 4-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate
N-{4-[(Z)-cyano(1-ethyl-1,3-dihydro-2H-benzimidazol-2-ylidene)methyl]-5-methylpyrimidin-2-yl}piperidine-4-carboxamide
tert-butyl [4-({4-[(Z)-cyano(1-ethyl-1,3-dihydro-2H-benzimidazol-2-ylidene)methyl]-5-methylpyrimidin-2-yl}amino)-4-oxobutyl]carbamate
N-4-[(Z)-cyano(1-ethyl-1,3-dihydro-2H-benzimidazol-2-ylidene)methyl]-5-methylpyrimidin-2-yl}cyclopentanecarboxamide
N-{4-[(Z)-cyano(1-ethyl-1,3-dihydro-2H-benzimidazol-2-ylidene)methyl]-5-methylpyrimidin-2-yl}-4-(4-methylpiperazin-1-yl)butanamide
N˜1˜-{4-[(Z)-cyano(1-ethyl-1,3-dihydro-2H-benzimidazol-2-ylidene)methyl]-5-methylpyrimidin-2-yl}-N˜3˜,N-3˜-dimethyl-beta-alaninamide
N-{4-[(Z)-cyano(1-ethyl-1,3-dihydro-2H-benzimidazol-2-ylidene)methyl]-5-methylpyrimidin-2-yl}-4-(dimethylamino)butanamide
(2Z)-(2-amino-5-methylpyrimidin-4-yl)(1-ethyl-1,3-dihydro-2H-benzimidazol-2-ylidene)acetonitrile
N-{4-[(Z)-cyano(1-ethyl-1,3-dihydro-2H-benzimidazol-2-ylidene)methyl]-5-methylpyrimidin-2-yl}-2-morpholin-4-ylacetamide
N˜2˜-benzyl-N˜1˜-{4-[(Z)-cyano(1-ethyl-1,3-dihydro-2H-benzimidazol-2-ylidene)methyl]-5-methylpyrimidin-2-yl}glycinamide
N-{4-[(Z)-cyano(1-ethyl-1,3-dihydro-2H-benzimidazol-2-ylidene)methyl]-5-methylpyrimidin-2-yl}-2-(1,1-dioxidothiomorpholin-4-yl)acetamide
N˜1˜-{4-[(Z)-cyano(1-ethyl-1,3-dihydro-2H-benzimidazol-2-ylidene)methyl]-5-methylpyrimidin-2-yl}-N˜2˜-formylglycinamide

Compounds of formula (I) are suitable for the use as medicament, in particular for the treatment and/or prevention of metabolic disorders mediated by insulin resistance or hyperglycemia, comprising diabetes type II, inadequate glucose tolerance, insulin resistance, obesity, polycystic ovary syndrome (PCOS).

The compounds according to formula I could be employed alone or in combination with further pharmaceutical agents.

In one embodiment, the compounds of formula (I) are useful in inhibiting Glycogen Synthase Kinase 3

Still a further object of the present invention is a process for preparing the benzimidazole acetonitriles according to formula I.

The benzimidazole acetonitriles exemplified in this invention may be prepared from readily available starting materials using the following general methods and procedures. It will be appreciated that where typical or preferred experimental conditions (i.e., reaction temperatures, time, moles of reagents, solvents, etc.) are given, other experimental conditions can also be used unless otherwise stated. Optimum reaction conditions may vary with the particular reactants or solvents used, but such conditions can be determined by one skilled in the art by routine optimisation procedures.

Generally, the benzimidazole acetonitriles derivatives according to the general formula I may be obtained by several processes using solution-phase chemistry protocols.

The general synthetic approach for obtaining compounds of formula (I) is depicted in Scheme 1. Therein, benzimidazole acetonitriles derivatives according to the general formula I, whereby the substituents L and G are as above defined, may be prepared from the corresponding acetonitrile derivatives IV and chloro derivatives V.

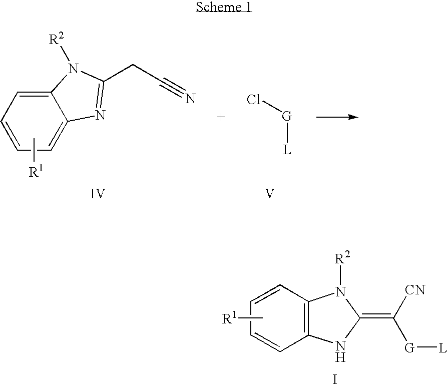

In a more specific method, the benzimidazole acetonitrile derivative VI with R¹ being as above defined—is reacted with the electrophile VII (e.g. alkyl chloride) to give the corresponding benzimidazole compounds IV. In a subsequent step, the intermediate IV is treated with a bis-chloro derivative V′, wherein G is as above defined, to give the intermediate of synthesis II. In a final step, the intermediate E may be treated with an amine III, whereby the substituents R⁵, R⁶ are as above defined to give the final benzimidazole acetonitrile derivatives I, utilizing well known solution-phase chemistry protocols, such as those described in the Examples and shown in Scheme 2, below:

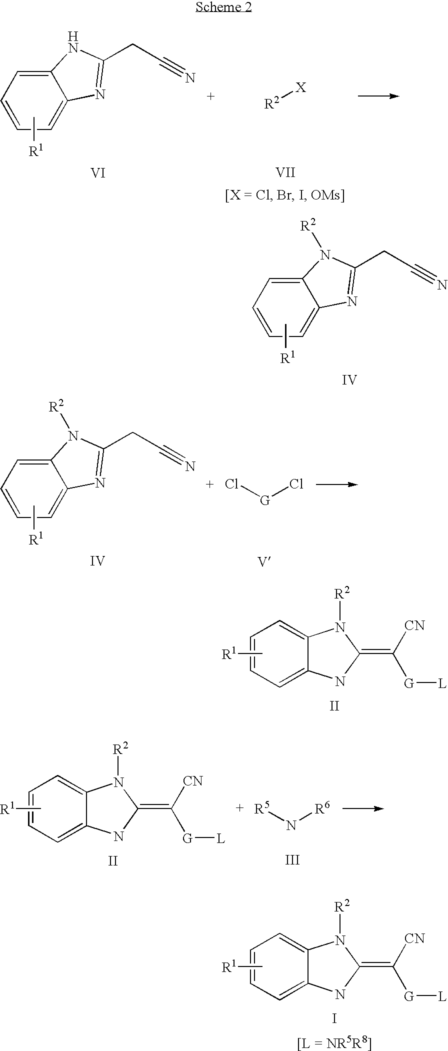

Electrophiles VII as well as bis-chloro derivative V′ and amines III are commercially available.

The benzimidazole acetonitriles derivatives according to the general formula I, may be obtained in 2-6 subsequent steps depending the availability of starting materials and building blocks. As shown in Scheme 3. In a first step, the benzimidazole acetonitriles derivatives IV are isolated after condensation of the benzimidazole compound VI with an electrophile VII, whereby R² is as above defined. Several reaction conditions may be utilised for performing this first reaction step, e.g. by the use of PS-TBD (7-methyl-1,5,7-triazabicyclo[4.4.0]dec-5-en′ on polystyrene HL) a polymer immobilised reagent as a base in presence of various electrophilic reactants such as alkyl chlorine, bromide, iodide or also activated alcohol through mesylate formation. This reaction may be performed in solvents like DCM or DCM/dioxane. This reaction can be performed at various temperature depending of the intrinsic reactivity of compounds VI and VII, by traditional thermal method, using standard conditions well known to the person skilled in the art, such as those described hereinafter in the Examples.

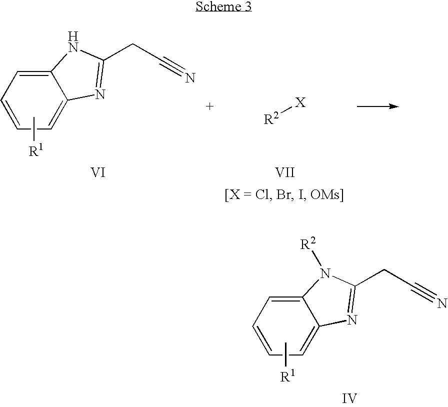

In a subsequent step, the benzimidazole acetonitriles derivatives II, whereby the substituents R¹ and R² are as above defined, are isolated after condensation of the benzimidazole compound IV with bis-chloro derivative V′. This reaction step is performed, using, e.g. lithium hydride or sodium hydride, cesium carbonate or similar reagents in an appropriate solvent such as Dioxane, TVF, DMA or DMF. This reaction can be performed at various temperature depending of the intrinsic reactivity of compounds IV and V′, by traditional thermal method or using microwave technology, using standard conditions well known to the person skilled in the art, such as those described hereinafter in the Examples.

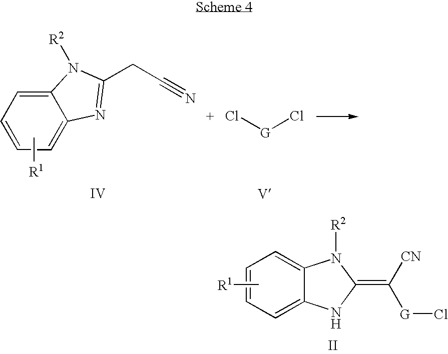

In a following step, as shown in Scheme 5, the chloro benzimidazole acetonitriles derivatives II may treated with various nucleophiles, e.g. an amine III, to give the expected benzimidazole acetonitriles I. The nucleophilic displacement of the chloro atom of the pyrimidinyl moiety by the amine III, is accomplished by treatment with several equivalents of the nucleophile, e.g. the amine III, in presence or absence of e.g. sodium iodine as catalyst and a base such as triethylamine of diisopropylethylamine or similar reagents. This reaction can be performed at various temperatures depending of the intrinsic reactivity of compounds II and III, by traditional thermal method or using microwave technology, using standard conditions well known to the person skilled in the art, such as those described hereinafter in the Examples.

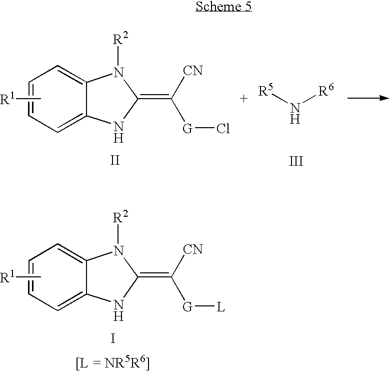

The benzimidazole acetonitriles derivatives according to the general formula I can be further isolated from the intermediate I′, whereby L is the moiety —NR⁵R⁶, with R⁵ being hydrogen and R⁶ is as above defined, as shown in Scheme 6. The benzimidazole derivatives I may be obtained by treatment of the intermediate I′ with either an acyl chloride, a carboxylic acid or a sulfonyl chloride using standard conditions well known to the person skilled in the art, such as amide bond formation protocols or sulfonamide formation using the appropriate reactants as those mentioned above and reagents such as bases like s triethylamine, pyridine etc, and activating agents e.g, HOBt, EDC or similar reagents in an appropriate solvent such as THF or DMF. This reaction can be performed at various temperature depending of the intrinsic reactivity of compounds Ib and VIII, by traditional thermal method or using microwave technology, using standard conditions well known to the person skilled in the art such as those described hereinafter in the Examples.

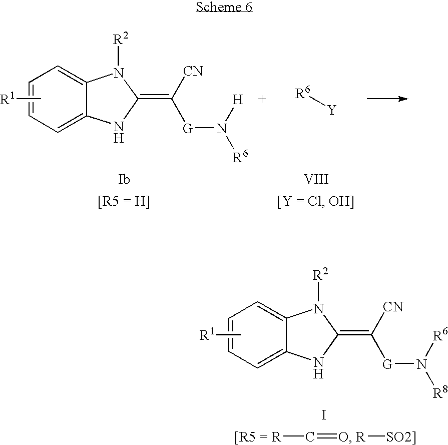

A specific functional moiety (R¹) may be converted into a different one (R^{1′}), using any known functional group interconversion protocols. As illustrated in Scheme 7, the choice of the best synthetic strategy will be governed by the nature of the functional groups to be interconverted, and the compatibility of the required reaction conditions with other functional groups present in the corresponding compounds, as will be well appreciated by the person skilled in the art. Amongst the most preferred starting materials I, II and IV and VII, are those wherein R¹ is —Br, —Cl, —I, —OH, —NH2, —CH₂OH, —CHO, —COOH, —NO₂, and/or —CH₂COOH, which are either obtained from commercial sources or made by one of the numerous processes described in the literature. From the intermediates (XXI, XXV, XXVII) derived thereof, in which R is as defined in Scheme 7, a wide range of derivatives, such as e.g. (XXII)-(XXXV), in which R⁹, R¹⁰, R¹¹, R⁷, are as defined below, can be obtained by reaction sequences including oxidations, reductions, O- and N-alkylations, reductive alkylations and aminations, chain-elongations, Mitsunobu reactions, acylation, debocylation, Wittig reactions, acylations, sulfonylations, Stille, Suzuki, Sonogashira and any other appropriate transformations leading to functional group interconversions, some of which being exemplified in Scheme 8. The synthetic examples cited in Scheme 8 are meant to illustrate the concept of functional group interconversion as applied to compounds of general structures (I), (II), (IV), and (VI), wherein R¹, R² are as defined in the above description and in Scheme 7, and are not construed to be viewed as limiting the scope of said synthetic approach.

R⁹, R¹⁰, R¹¹, R⁷, are each independently from each other H, unsubstituted or substituted C₁-C₆-alkyl, unsubstituted or substituted C₂-C₆-alkenyl, unsubstituted or substituted C₂-C₆-alkynyl, unsubstituted or substituted C₁C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), unsubstituted or substituted C₁-C₆-alkyl aryl, unsubstituted or substituted C₁-C₆-alkyl heteroaryl, usubstituted or substituted C₁-C₆-alkenyl aryl, unsubstituted or substituted C₁-C₆-alkenyl heteroaryl, unsubstituted or substituted C₁-C₆-alkynyl aryl, unsubstituted or substituted C₁-C₆-alkynyl heteroaryl, unsubstituted or substituted C₁-C₆-allyl cycloalkyl, unsubstituted or substituted C₁-C₆-alkyl heterocycloalkyl, unsubstituted or substituted C₁-C₆-alkenyl cycloalkyl, unsubstituted or substituted C₁-C₆-alkenyl heterocycloalkyl, unsubstituted or substituted C₁-C₆-alkynyl cycloalkyl, unsubstituted or substituted C₁-C₆-alkynyl heterocycloalkyl.

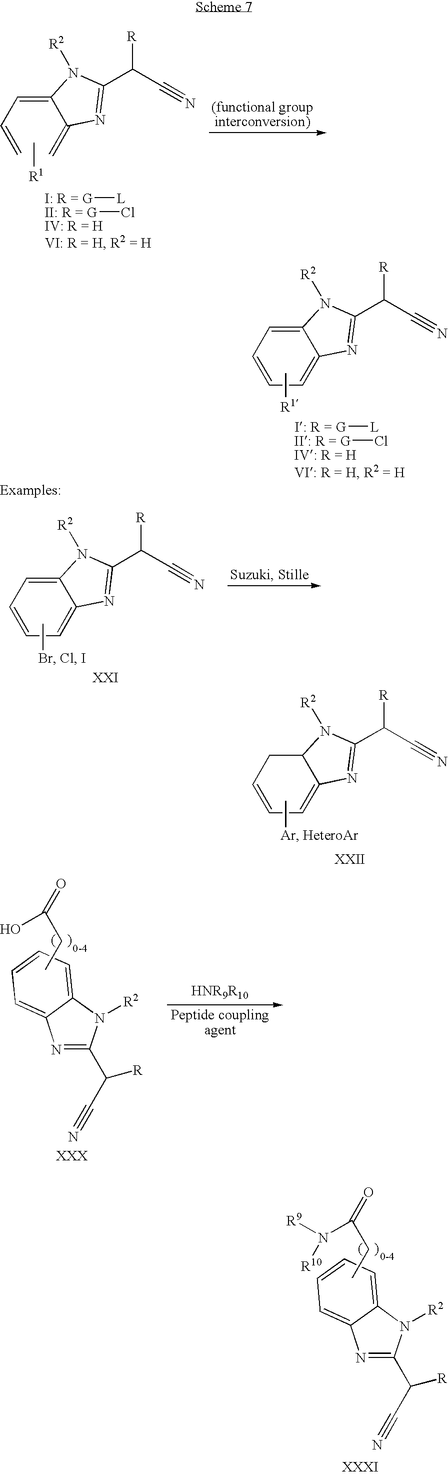

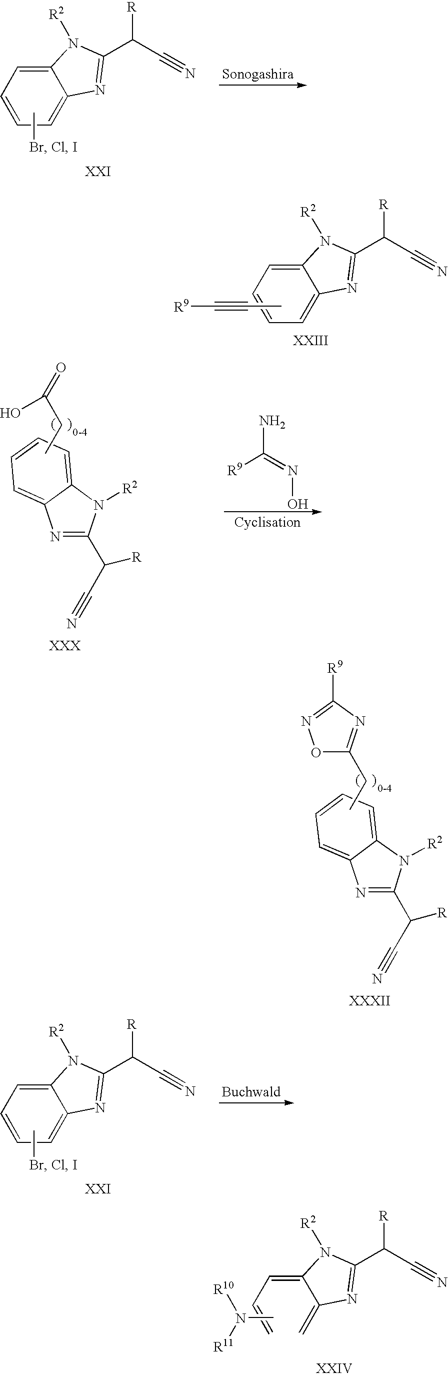

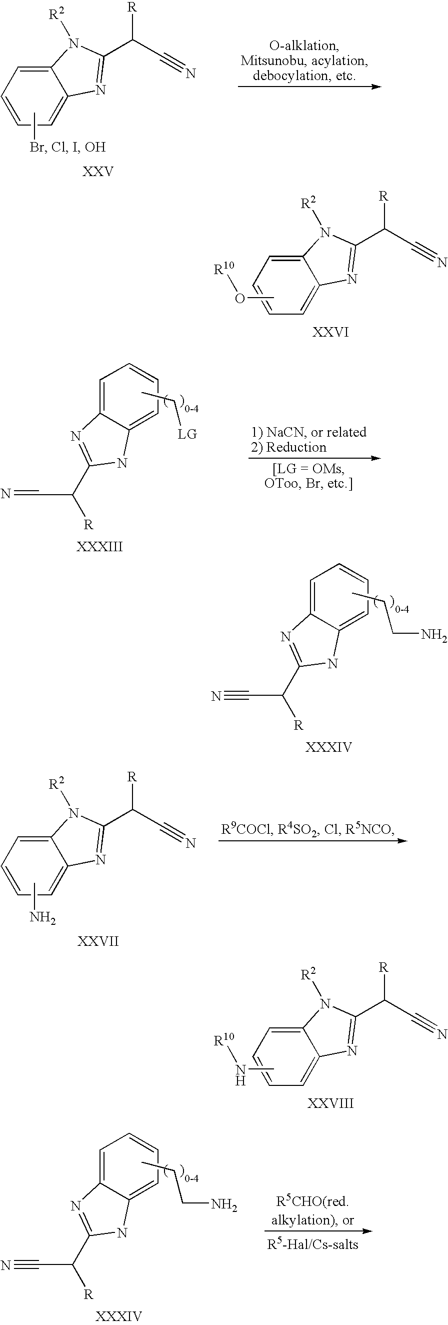

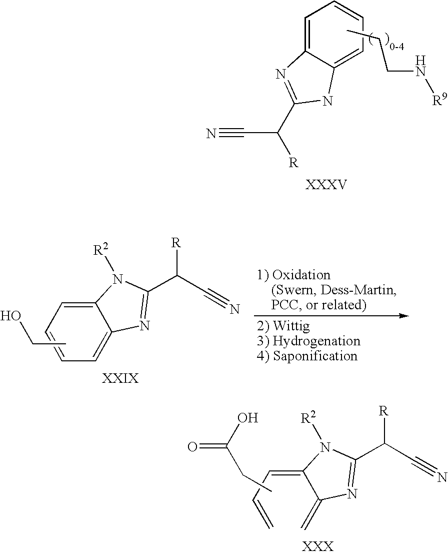

The benzimidazole acetonitriles derivatives according to the general formula I, may be obtained in 2-6 subsequent steps depending the availability of starting materials and building blocks. As shown in Scheme 9. In a first step, the benzimidazole acetonitriles derivatives II′ are isolated after condensation of the benzimidazole compound II with a solution of ammonium hydroxide. This reaction may be performed in solvents like DMA, isopropanol or solution containing both solvents in various ratio. This reaction can be performed at various temperature depending of the intrinsic reactivity of compounds II, by traditional thermal method or using microwave technology, using standard conditions well known to the person skilled in the art, such as those described hereinafter in the Examples

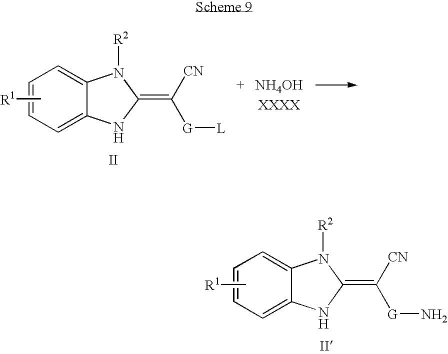

In a following step, the benzimidazole acetonitriles derivatives according to the general formula I can be further isolated from the intermediate III′, whereby L is the moiety—C(O)R⁶, with R⁶ is as above defined, as shown in Scheme 10. The benzimidazole derivatives I may be obtained by treatment of the intermediate II′ with either an acyl chloride or a carboxylic acid using standard conditions well known to the person skilled in the art, such as amide bond formation protocols using the appropriate reactants as those mentioned above and reagents such as bases like triethylamine, pyridine etc, and activating agents e.g, HOBt, EDC, Mukayama reagent or similar reagents in an appropriate solvent such as DCM, THF or DMF. This reaction can be performed at various temperature depending of the intrinsic reactivity of compounds II′ and III′, by traditional thermal method or using microwave technology, using standard conditions well known to the person skilled in the art, such as those described hereinafter in the Examples.

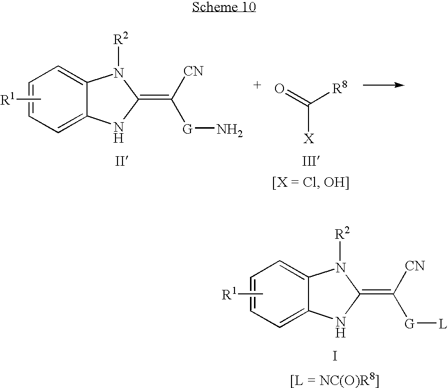

When employed as pharmaceuticals, the benzimidazole acetonitriles of the present invention are typically administered in the form of a pharmaceutical composition. Hence, pharmaceutical compositions comprising a compound of formula (I) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

When employed as pharmaceuticals, benzimidazole acetonitriles of this invention are typically administered in the form of a pharmaceutical composition Such compositions can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, intrathecal, intraperitoneal and intranasal. Depending on the intended route of delivery, the compounds are preferably formulated as either injectable, topical or oral compositions. The compositions for oral administration may take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the benzimidazole acetonitrile compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As mentioned above, the benzimidazole acetonitriles of formula I in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein be reference.

The compounds of this invention can also be administered in sustained release forms or from sustained release drug delivery systems. A description of representative sustained release materials can also be found in the incorporated materials in Remington's Pharmaceutical Sciences.

A further aspect of the present invention is related to a pharmaceutical composition composition a comprising a benzimidazole derivative according to formula (I) and at least one further drug (in particular an anti-diabetes agent). In one embodiment the further diabetes agents are selected from the group comprising or consisting of insulin (or insulin mimicks), aldose reductase inhibitors, alpha-glucosidase inhibitors, sulfonyl urea agents, biguanides (e.g. metformin), thiazolidines (e.g. pioglitizone, rosiglitazone, cf. WO 02/100396), a PTP1B inhibitor, a PPAR agonists or a GSK-3 inhibitor.

Insulins useful with the method of the present invention include rapid acting insulins, intermediate acting insulins, long acting insulins and combination of intermediate and rapid acting insulins.

Among the more preferred aldose reductase inhibitors of this invention are minalrestat, Tolrestat, Sorbinil, Methosorbinil, Zopolrestat, Epalrestat, Zenarestat, Imirestat and Ponalrestat or the pharmaceutically acceptable salt forms thereof.

The alpha-glucosidase inhibitors useful for the method of the present invention include miglitol or acarbose, or the pharmaceutically acceptable salt form thereof.

Sulfonylurea agents useful with the method of the present invention include glipizide, Glyburide (Glibenclamide), Clorpropamide, Tolbutamide, Tolazamide and Glimepiride, or the pharmaceutically acceptable salt forms thereof.

Preferably, said supplementary pharmaceutically active agent is selected from the group consisting of a rapid acting insulin, an intermediate acting insulin, a long acting insulin, a combination of intermediate and rapid acting insulins, Inalrestat, Tolrestat, Sorbinil, Methosorbinil, Zopolrestat, Epalrestat, Zenarestat, Imirestat, Ponalrestat, ONO-2235, GP-1447, CT-112, BAL-ARI 8, AD-5467, ZD5522, M-16209, NZ-314, M-79175, SPR-210, ADN 138, or SNK-860, Miglitol, Acarbose, Glipizide, Glyburide, Chlorpropamide, Tolbutamide, Tolazamide, or Glimepriride.

In the following the present invention shall be illustrated by means of some examples which are not construed to be viewed as limiting the scope of the invention.

The following abbreviations are hereinafter used in the accompanying examples: min (minute), hr (hour), g (gram), mmol (millimole), m.p. (melting point), eq (equivalents), mL (milliliter), μL (microliters), mL (milliliters), ACN (Acetonitrile), Boc (butoxycarbonyl), CDCl₃ (deuterated chloroform), CsCO₃ (Cesium carbonate), cHex (Cyclohexanes), DCM (Dichloromethane), DIC (Diisopropyl carbodiimide), DIPEA (Diisopropylamine), DMA (Dimethylacetamide), DMAP (4-Dimethylaminopyridine) DMF (Dimethylformamide), DMSO (Dimethyl-sulfoxide), DMSO-d₆ (deuterated dimethylsulfoxide), EDC (1-(3-Dimethyl-amino-propyl)-3-ethylcarbodiimide), Et₃N (Triethylamine), EtOAc (Ethyl acetate), EtOH (Ethanol), Et₂O (Diethyl ether), Fmoc (9-fluorenyl-methoxycarbonyl), HOBt (1-Hydroxybenzotriazole), iPrOH (Isopropanol), K₂CO₃ (potassium carbonate), LiH (Lithium Hydride), NaI (Sodium Iodine), NaH (Sodium hydride), NaHCO₃ (Sodium bicarbonate), NH₄Cl (Ammonium chloride), nBuLi (n Butyllithium), Pd(PPh₃)₄ (Palladium triphenylphosphine tetrakis), (TBTU (O-Benzotriazolyl-N,N,N′,N′-tetramethyluronium-tetrafluoroborate), Mukayama reagent (1-methyl-2-chloropyridinium iodide), TEA (Triethyl amine), TFA (Trifluoro-acetic acid), THF (Tetrahydrofuran), TBD-resin (7-methyl-1,5,7-triazabicyclo[4.4.0]dec-5-ene on polystyrene.HL), TMOF (trimethylorthoformate), MgSO₄ (Magnesium sulfate), PetEther (Petroleum ether), rt (room temperature).

The HPLC, NMR and MS data provided in the examples described below were obtained as followed: HPLC: column Waters Symmetry C8 50×4.6 mm, Conditions: MeCN/H₂O, 5 to 100% (8 min), max plot 230-400 nm; Mass spectra: PE-SCIEX API 150 EX (APCI and ESI), LC/MS spectra: Waters ZMD (ES); ¹H-NMR: Bruker DPX-300 MHz. The purifications were obtained as followed: Preparative HPLC Waters Prep LC 4000 System equipped with columns Prep Nova-Pak®HR C 186 μm 60 Å, 40×30 mm (up to 100 mg) or 40×300 mm (up to 1 g). All the purifications were performed with a gradient of MeCN/H₂O 0.09% TFA.

### EXAMPLES

### Intermediate 1: (1-ethyl-2,3-dihydro-1H-benzimidazol-2-yl)acetonitrile (cf. Scheme 4, compound IV)

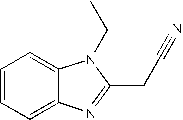

To a suspension of 2-benzimidazolylacetonitrile (6.0 g, 38.17 mmol) in DCM (200 mL) was added TBD-resin (17.6 g, 45.81 mmol, loading 2.6 mmol/g) followed by bromoethane (4.08 mL, 38.17 mmol) at room temperature. The reaction mixture was shaken for 4 days at room temperature, then the resin was filtered and washed with 50 mL of DCM. The filtrate was concentrated to give the pure (1-ethyl-2,3-dihydro-1H-benzimidazol-2-yl)acetonitrile as an yellow-orange solid (5.25 g, 74.2% yield, 98% HPLC purity). This compound was utilised as such for the next reaction.

1H NMR (300 MHz, CDCl3); 1.5 (t, 3H), 4.0 (s, 2H), 4.2 (q, J=7.5 Hz, 2H), 7.2-7.45 (m, 3H), 7.7 (d, J=7.0 Hz, 1H). MS(ESI⁺): 188.4; MS(ESI⁻): 186.5.

### Intermediate 2: (1-propyl-2,3-dihydro-1H-benzimidazol-2-yl)acetonitrile (cf. Scheme 4, compound IV)

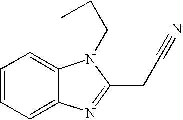

Following the general methods as outlined in Intermediate 1, starting from 2-benzimidazolylacetonitrile and 1-bromopropane, the title compound was isolated, after filtration and evaporation, as an orange solid in 49% yield (98.5% purity by HPLC).

1H NMR (300 MHz, CDCl3); 1.5 (t, 3H), 1.6 (m, 2H), 4.0 (s, 2H), 4.1 (m, 2H), 7.3-7.45 (m, 3H), 7.6 (d, J=7.0 Hz, 1H). MS(ESI⁺): 202.3; MS(ESI⁻): 200.4.

### Intermediate 3: (1-cyclobutyl-2,3-dihydro-(1H-benzimidazol-2-yl)acetonitrile (cf. Scheme 4, compound IV)

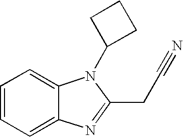

Following the general methods as outlined in Intermediate 1, starting from 2-benzimidazolylacetonitrile and bromocyclobutane, the title compound was isolated, after filtration and evaporation, as an orange solid in 22% yield (97% purity by HPLC).

1H NMR (300 MHz, CDCl3); 1.6-1.9 (m, 6H), 4.1 (s, 2H), 4.8 (m, 1H), 7.3-7.5 (m, 3H), 7.6 (d, J=7.0 Hz, 1H). MS(ESI⁺): 214.3; MS(ESI⁻): 212.4.

### Intermediate 6: (2-chloro-5-methylpyrimidin-4-yl)(1-ethyl-2,3-dihydro-1H-benzimidazol-2-yl)acetonitrile (cf Scheme 9, compound V′c)

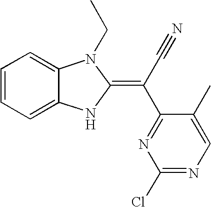 Method A:

To a suspension of Cesium carbonate (13.85 g, 42.55 mmol) in dioxane (50 mL), was added a solution of (1-ethyl-2,3-dihydro-1H-benzimidazol-2-yl)acetonitrile (Intermediate 1, 5.25 g, 28.34 mmol) in dioxane (50 mL). The reaction mixture was stirred at room temperature for 3 hours. A solution of 2,4-dichloro-5-methyl-pyrimidine (5.54 g, 34.01 mmol) in dioxane (100 ml), was added dropwise at room temperature. The reaction mixture was stirred and heated to reflux for 24 hours. The reaction mixture was allowed to warm to r.t. and water was added (50 mL). The solvents were concentrated under vacuum to 50 mL and a solution of 1N HCl (50 mL) was added at zero degree. The solution was then diluted to ⅓ with acetonitrile and concentrated under vacuum to 50 mL. The precipitate was filtered and washed with water (10 mL), acetonitrile (10 mL) and diethyl ether (10 mL) to give a yellow solid which was dried under vacuum. The yellow crystalline product (2-chloro-5-methylpyrimidin-4yl)(1-ethyl-2,3-dihydro-1H-benzimidazol-2-yl)acetonitrile was isolated in 95% HPLC purity (Yield 5.1 g, 50%).

Method B:

To a suspension of Cesium carbonate (96 mg, 0.3 mmol) in dioxane (2 mL), was added a solution of (1-ethyl-2,3-dihydro-1H-benzimidazol-2-yl)acetonitrile (Intermediate 1, 50 mg, 0.27mmol) in dioxane (2 mL). Then a solution of 2,4-dichloro-5-methyl-pyrimidine (57 mg, 0.37 mmol) in dioxane (1 ml), was added at room temperature. The reaction mixture was stirred and heated to 160 degrees in the microwave for 25 minutes. The reaction mixture was allowed to warm to r.t. and water was added (1 mL). The solvents were concentrated under vacuum to 5 mL and a solution of 1N HCl (5 mL) was added at zero degree. The solution was then diluted to ⅓ with acetonitrile and concentrated under vacuum to 5 mL. The precipitate was filtered and washed with water (2 mL), acetonitrile (2 mL) and diethyl ether (2 mL) to give a yellow solid which was dried under vacuum. The yellow crystalline product (2-chloro-5-methylpyrimidin-4-yl)(1-ethyl-2,3-dihydro-1H-benzimidazol-2-yl)acetonitrile was isolated in 92% HPLC purity (Yield 45 mg, 53%).

(2-chloro-5-methylpyrimidin-4-yl)(1-ethyl-2,3-dihydro-1H-benzimidazol-2-yl)acetonitrile: 1H NMR (300 MHz, CDCl3); 1.6 (t, J=7.5 Hz, 3H), 4.7 (q, J=7.5 Hz, 2H), 2.3 (s, 3H), 7.2-7.45 (m, 4H), 8.2 (d, J=7.0 Hz, 1H), 13.7 (s(broad), 1H). MS(ESI³⁰ ): 314.8; MS(ESI⁻): 312.6.

### Intermediate 7: (2-chloropyrimidin-4-yl)(1-ethyl-2,3-dihydro-1H-benzimidazol-2-yl)acetonitrile (cf Scheme 5, compound II)

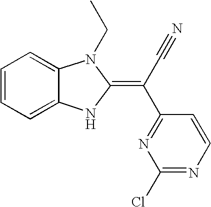

Following the general method A as outlined in Intermediate 6, starting from (1-ethyl-2,3-dihydro-1H-benzimidazol-2-yl)acetonitrile (Intermediate 1) and 2,4-dichloropyrimidine, the title compound was isolated, as a yellow solid in 51% yield (96% purity by HPLC).

1H NMR (300 MHz, CDCl3); 1.6 (t, J=7.5 Hz, 3H), 4.7 (q, J=7.5Hz, 2H), 7.2-7.5 (m, 5H), 8.2 (d, J=7.0 Hz, 1H), 13.7 (s(broad), 1H). MS(ESI⁺): 300.8; MS(ESI⁻): 298.8.

### Intermediate 8: (2-chloro-5-methylpyrimidin-4-yl(2,3-dihydro-1H-benzimidazol-2-yl)acetonitrile (cf. Scheme 5, compound II)

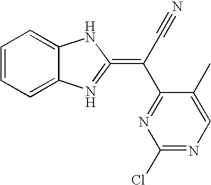

Following the general method A as outlined in Intermediate 6, starting from 2-benzimidazolylacetonitrile and 2,4-dichloro-5-methyl-pyrimidine, the title compound was isolated, as a yellow solid in 60% yield (95% purity by HPLC).

MS(ESI⁺): 286.8 (ESI⁻): 284.7.

### Intermediate 9: (2-chloropyrimidin-4-yl)(2,3-dihydro-1H-benzimidazol-2-yl)acetonitrile (cf. Scheme 5, compound II)

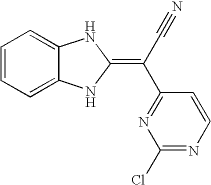

Following the general method B as outlined in Intermediate 6, starting from 2-benzimidazolylacetonitrile and 2,4-dichloro-pyrimidine, the title compound was isolated, as a yellow solid in 55% yield (94% purity by HPLC).

MS(ESI⁺): 272.7 (ESI⁻): 270.6.

### Intermediate 10: (2-chloro-6-methylpyrimidin-4-yl)(1-ethyl-2,3-dihydro-1H-benzimidazol-2-yl)acetonitrile (cf. Scheme 5, compound II)

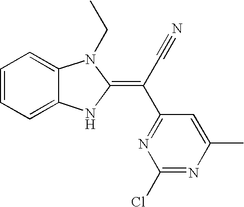

Following the general method A as outlined in Intermediate 6, starting from (1-ethyl-2,3-dihydro-1H-benzimidazol-2-yl)acetonitrile (Intermediate 1) and 2,4-dichloro-6-methyl-pyrimidine, the title compound was isolated, as a yellow solid in 49% yield (97% purity by HPLC).

MS(ESI⁺): 314.9 (ESI⁻): 312.7.

### Intermediate 11: (6-chloropyrimidin-4-yl)(1-ethyl-2,3-dihydro-1H-benzimidazol-2-yl)acetonitrile (cf. Scheme 5, compound II)

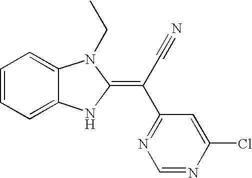

Following the general method A as outlined in Intermediate 6, starting from (1-ethyl-2,3-dihydro-1H-benzimidazol-2-yl)acetonitrile (Intermediate 1) and 4,6-dichloropyrimidine, the title compound was isolated, as a yellow solid in 46% yield (92% purity by HPLC).

MS(ESI⁺): 300.7; MS(ESI⁻): 298.4.

### Intermediate 15: (2-chloro-5-methylpyrimidin-4-yl)(1,3-diethyl-2,3-dihydro-1H-benzimidazol-2-yl)acetonitrile (cf. Scheme 5, compound II)

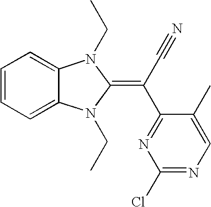

Following the general method A as outlined in Intermediate 6, starting from (1,3-diethyl-benzimidazol-2-yl)acetonitrile and 2,4-dichloro-5-methyl-pyrimidine, the title compound was isolated, as a yellow solid in 39% yield (94% purity by HPLC).

MS(ESI⁺): 342.9; MS(ESI⁻): 340.8.

### Intermediate 17: (2-chloropyrimidin-4-yl)(1-propyl-2,3-dihydro-1H-benzimidazol-2-yl)acetonitrile (cf. Scheme 5, compound II)

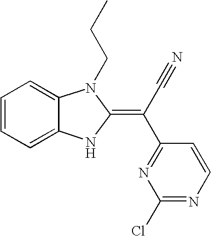

Following the general method A as outlined in Intermediate 6, starting from from (1-propyl-2,3-dihydro-1H-benzimidazol-2-yl)acetonitrile (intermediate 2) and 2,4-dichloro-pyrimidine, the title compound was isolated, as a yellow solid in 66% yield (99% purity by HPLC).

MS(ESI⁺): 314.8; MS(ESI⁻): 312.6.

### Intermediate 18: (2-chloropyrimidin-4-yl)(1-cyclobutyl-2,3-dihydro-1H-benzimidazol-2-yl)acetonitrile (cf. Scheme 5, compound II)

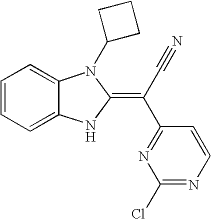

Following the general method A as outlined in Intermediate 6, starting from from (1-cyclobutyl-2,3-dihydro-1H-benzimidazol-2-yl)acetonitrile (Intermediate 3) and 2,4-dichloro-pyrimidine, the title compound was isolated, as a yellow solid in 50% yield (99% purity by HPLC).

MS(ESI⁺): 326.9; MS(ESI⁻): 324.8.

### Intermediate 19: (2Z)-(2-amino-5-methylpyrimidin-4-yl)(1-ethyl-1,3-dihydro-2H-benzimidazol-2-ylidene)acetonitrile (cf. Scheme 9, compound II′)

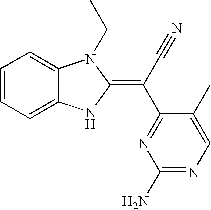

To a solution of (2-chloro-5-methylpyrimidin-4-yl)(1-ethyl-2,3-dihydro-1H-benzimidazol-2-yl)acetonitrile (Intermediate 6, 1.5 g, 5.05 mmol) in iPrOH (2 mL), were added ammonium hydroxide (3.14 mL, 81.59 mmol, 16 eq) and sodium iodide (378 mg, 2.52 mmol, 0.5 eq). The reaction mixture was heated up to 160 degree for 60 min in a microwave device. After completion of the reaction, the reaction mixture was cooled down to room temperature and the yellow precipitate was filtered off, washed with water and dried under vacuum overnight to give the expected compound (2Z)-(2-amino-5-methylpyrimidin-4-yl)(1-ethyl-1,3-dihydro-2H-benzimidazol-2-ylidene)acetonitrile as a yellow solid (1.41 mg, 82% yield, 95% HIPLC purity)

(2Z)-(2-amino-5-methylpyrimidin-4-yl)(1-ethyl-1,3-dihydro-2H-benzimidazol-2-ylidene)acetonitrile: 1H NMR (300 MHz, CDCl3); 1.55 (t, J=7.4 Hz, 3H), 4.6 (q, J=7.4 Hz, 2H), 2.4 (s, 3H), 7.2-7.5 (m, 4H), 8.2 (d, J=7.0 Hz, 1H), 14.1 (s(broad), 1H). MS(ESI⁺): 293.5; MS(ESI⁻): 291.4.

### Intermediate 20: (2Z)-(2-amino-pyrimidin-4-yl)(1-ethyl-1,3-dihydro-2H-benzimidazol-2-ylidene)acetonitrile (cf. Scheme 9, compound II′)

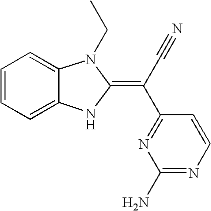

Following the general method as outlined in Intermediate 19, starting from (2-chloropyrimidin-4-yl)(1-ethyl-2,3-dihydro-1H-benzimidazol-2-yl)acetonitrile (Intermediate 7) and ammonium hydroxide, the title compound was isolated, as a yellow solid in 75% yield (96% purity by HPLC).

MS(ESI⁺): 279.51 MS(ESI⁻): 277.4.

### Example 1

### General procedure for the solution-phase synthesis of benzimidazoles acetonitriles derivatives of general formula I, with G and L as above defined (Schemes 1-6): (2Z)-(1-ethyl-1,3-dihydro-2H-benzimidazol-2-ylidene)(2-{[3-(1H-pyrazol-1-yl)propyl]amino}pyrimidin-4-yl)acetonitrile

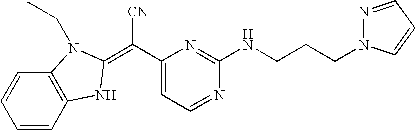 Method C:

To a solution of (2-chloropyrimidin-4-yl)(1-ethyl-2,3-dihydro-1H-benzimidazol-2-yl)acetonitrile (intermediate 7) (150 mg, 0.5 mmol, 1 eq) in 4 mL of DMA:iPrOH (1:3)) was added DIEA (0.345 mL, 2.0 mmol, 4 eq), 3-((1H-pyrazol-1-yl)propan-1-amine hydrochloride (325 mg, 2.0 mmol, 4 eq) and NaI (38 mg, 0.25 mmol, 0.5 eq). The reaction mixture was heated up to 180° C. for 2.5 hours in a microwave device. The isopropanol was evaporated and the residue redissolved in 3mL of DGM This solution was loaded onto a 10 g SCX-SPE syringe (0.4 mmol·g⁻¹) and eluted with DCM, then DCM:MeOH (1:1), 0.1 μM NH₃ in MeOH and 1M NH₃ in MeOH. The 4 fraction were analyzed by HPLC and LC-MS and the fractions contained the product were mixed together. The solvents were evaporated and the residue redissolved in DCM and washed with NaHCO3 sat. and brine. The organic layer was dried over MgSO₄, filtered and the solvent evaporated. The residue was then purified by preparative HPLC with a gradient 10 to 100& acetonitrile in 0.1M TFΛ. The solution was evaporated and the desired compound as a TFA salt, was isolated as a yellow solid (180 mg, 0.36 mmol, yield: 72%, 990A HPLC purity).

Method D:

10 mg of Building Blocks were dissolved in 0.3 mL of DMA. Et₃N (4 eq.) and the amines (4 eq.)dissolved in DMA (0.3 mL) were then added to the reaction mixtures and the plate was sealed and heated in a microwave (Mars 5) as follow: 2 plates at a time were heated 4 min at 300 Watts and then left to cool down for 10 min. This was repeated 4 times. The reaction mixtures were then transferred into a 2 mL plate and the solvent was removed in the Genevac. Work up: 1 mL of water/CH₃COOH (2%) was then added and the plate was shaken for 3 h00. The aqueous layer was removed using the Zymark, leaving the solid behind. This solid was further washed with water (twice). 1 mL of MeOH/TFA (20%) was added to the plates, which were shaken at room temperature for 48 h00 and the supernatant was collected using the Lissy. Analytical plates were made and the solvents were removed in the Genevac.

(2Z)-(1-ethyl-1,3-dihydro-2H-benzimidazol-2-ylidene)(2-{[3-(1H-pyrazol-1-yl)propyl]amino}pyrimidin-4-yl)acetonitrile: yellow solid; ¹H NMR (300 MHz, CDCl₃); 1.5 (t, 3H), 2.6-2.8 (m, 2H), 3.5-3.7 (m, 2H), 4.4-4.6 (m, 2H), 4.7-4.85 (q, 2H), 6.8-6.9 (m, 1H), 7.4-7.7 (m, 3H), 7.75 (m, 1H), 7.85 (m, 1H), 10.1 (m, 1H). MS(ESI+), 387.5, (ESI−), 385.6.

### Example 2

### (2Z)-(1-ethyl-1,3-dihydro-2H-benzimidazol-2-ylidene)(5-methyl-2-{[3-(1H-pyrazol-1-yl)propyl]amino}pyrimidin-4-yl)acetonitrile

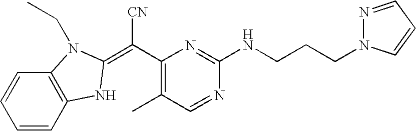

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and 3-((1H-pyrazol-1-yl)propan-1-amine hydrochloride, the title compound was isolated, as a yellow solid in 82% yield (99% purity by HPLC).

MS(ESI⁺): 401.5; MS(ESI⁻): 399.2.

### Example 3

### (2Z)-[2-cyclobutylamino)-5-methylpyrimidin-4-yl](1-ethyl-1,3-dihydro-2H-benzimidazol-2-ylidene)acetonitrile

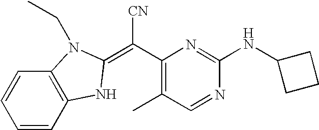

Following the general methods as outlined in Example 1 (Method C), starting from (2-chloro-5-methylpyrimidin-4-yl)(1-ethyl-2,3-dihydro-(1H-benzimidazol-2-yl)acetonitrile (intermediate 6), and cyclobutylamine, the title compound was isolated, as a yellow solid in 78% yield (99% purity by HPLC).

¹H NMR (300 MHz, CDCl₃); 1.5 (t, 3H), 1.8-2.45 (m, 6H), 2.3 (s, 3H), 4.1-4.3 (m, 1H), 4.5-4.65 (q, 2H), 7.4-7.65 (m, 3H), 7.75-7.85 (m, 1H), 9.55 (m, 1H). MS(ESI⁺): 347.6; MS(ESI⁻): 345.3.

### Example 4

### (2Z)-(1-ethyl-1,3-dihydro-2H-benzimidazol-2-ylidene)(5-methyl-2-{[3-(2-oxopyrrolidin-1-yl)propyl]amino}pyrimidin-4-yl)acetonitrile

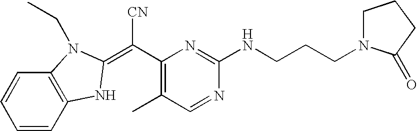

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and N-(3′-aminopropyl)-2-pyrrolidinone, the title compound was isolated, as a yellow solid in 77% yield (99% purity by HPLC).

¹H NMR (300 MHz, CDCl₃); 1.5 (t, 3H), 1.75-1.95 (m, 4H), 2.1-2.25 (m, 2H), 2.29 (s, 3H), 3.3-3.45 (m, 6H), 4.4-4.6 (q, 2H), 7.2-7.3 (m, 1H), 7.4-7.65 (m, 3H), 7.75-7.85 (m, 1H), 8.3 (m, 1H). MS(ESI⁺): 418.6; MS(ESI⁻): 416.8.

### Example 5

### (2Z)-(1-ethyl-1,3-dihydro-2H-benzimidazol-2-ylidene)(2-{[3-(2-oxopyrrolidin-1-yl)propyl]amino}pyrimidin-4-yl)acetonitrile

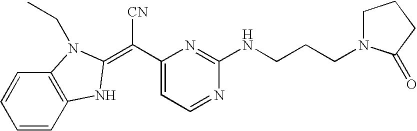

Following the general methods as outlined in Example 1 (Method C), starting from (2-chloropyrimidin-4-yl)(1-ethyl-2,3-dihydro-1H-benzimidazol-2-yl)acetonitrile (intermediate 7), and N-(3′-aminopropyl)-2-pyrrolidinone, the title compound was isolated, as a yellow solid in 70% yield (98% purity by HPLC).

MS(ESI⁺): 404.6; MS(ESI⁻): 402.8.

### Example 6

### (2Z)-(1-ethyl-1,3-dihydro-2H-benzimidazol-2-ylidene)(2-{[3-(1H-1,2,4-triazol-1-yl)propyl]amino}pyrimidinyl)acetonitrile

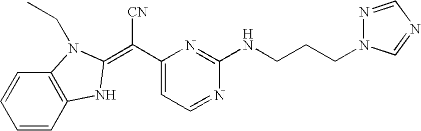

Following the general methods as outlined in Example 1 (Method C), starting from (2-chloropyrimidin-4yl)(1-ethyl-2,3-dihydro-(1H-benzimidazol-2-yl)acetonitrile (intermediate 7), and 3-(1H-1,2,4-triazol-1-yl)propan-1-amine hydrochloride, the title compound was isolated, as a yellow solid in 72% yield (98% purity by HPLC).

MS(ESI⁺): 388.7; MS(ESI⁻): 386.5.

### Example 7

### (2Z)-(1-ethyl-1,3-dihydro-2H-benzimidazol-2-ylidene)(5-methyl-2-{[3-(1H-1,2,4-triazol-1-yl)propyl]amino}pyrimidin-4-yl)acetonitrile

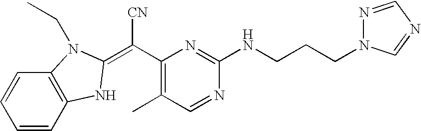

Following the general methods as outlined in Example 1 (Method C), starting from (2-chloro-5-methylpyrimidin-4-yl)(1-ethyl-2,3-dihydro-(1H-benzimidazol-2-yl)acetonitrile (intermediate 6), and 3-(1H-1,2,4-triazol-1-yl)propan-1-amine hydrochloride, the title compound was isolated, as a yellow solid in 78% yield (98% purity by “PLC).

¹H NMR (300 MHz, CDCl₃); 1.55 (t, 3H), 2.2-2.35 (m, 2H), 2.4 (s, 3H), 3.4-3.6 (m, 2H), 4.3-4.45 (m, 2H), 4.6-4.75 (q, 2H), 7.2-7.3 (m, 1H), 7.4-7.65 (m, 3), 7.75-7.85 (m, 1H), 7.9 (s, 1H), 8.3 (m, 1H), 9.8 (s, 1H). MS(ESI⁺): 402.6; MS(ESI⁻): 400.2.

### Example 8

### [2-(cyclopentylamino)-5-methylpyrimidin-4-y](1-ethyl-1H-benzimidazol-2-yl)acetonitrile

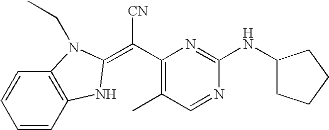

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and cyclopentylamine, the title compound was isolated, as a yellow solid in 690/o yield (99% purity by HPLC).

MS(ESI⁺): 361.4; MS(ESI⁻): 359.5.

### Example 9

### (2Z)-(2-{[3-(2-oxopyrrolidin-1-yl)propyl]amino}pyrimidin-4-yl)(1-propyl-1,3-dihydro-2H-benzimidazol-2-ylidene)acetonitrile

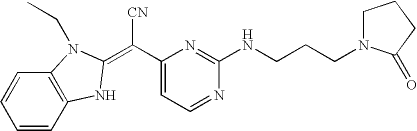

Following the general methods as outlined in Example 1 (Method C), starting (2-chloropyrimidin-4-yl)(1-propyl-2,3-dihydro-1H-benzimidazol-2-yl)acetonitrile (intermediate 17), and N-(3′-aminopropyl)-2-pyrrolidinone, the title compound was isolated, as a yellow solid in 79% yield (99% purity by HPLC).

MS(ESI⁺): 418.6; MS(ESI⁻): 416.5.

### Example 10

### (1-ethyl-1H-benzimidazol-2-yl){5-methyl-2-[(2-pyridin-3-ylethyl)amino]-pyrimidin-4-yl}acetonitrile

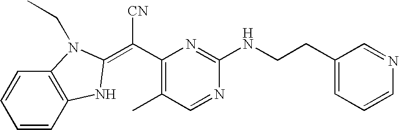

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and 3-(2-aminoethyl)pyridine, the title compound was isolated, as a yellow solid in 69% yield (96% purity by HPLC).

MS(ESI⁺): 398.6; MS(ESI⁻): 396.4.

### Example 11

### (2Z)-[2-(cyclobutylamino)pyrimidin-4-yl](1ethyl-1,3-dihydro-2H-benzimidazol-2-ylidene)acetonitrile

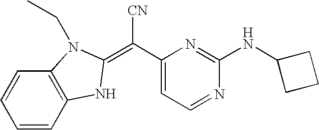

Following the general methods as outlined in Example 1 (Method C), (2-chloropyrimidin-4-yl)(1-ethyl-2,3-dihydro-1H-benzimidazol-2-yl)acetonitrile (intermediate 7), and cyclobutylamine, the title compound was isolated, as a yellow solid in 80% yield (97% purity by HPLC).

MS(ESI⁺): 333.2; MS(ESI⁻): 331.6.

### Example 12

### (2Z)-[2-(cycloheptylamino)-5-methylpyrimidin-4-yl](1-ethyl-1,3-dihydro-2H-benzimidazol-2-ylidene)acetonitrile

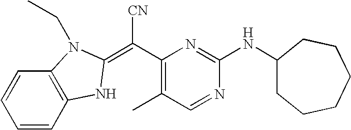

Following the general methods as outlined in Example 1 (Method C), starting from (2-chloro-5-methylpyrimidin-4-yl)(1-ethyl-2,3-dihydro-(1H-benzimidazol-2-yl)acetonitrile (intermediate 6), and cycloheptylamine, the title compound was isolated, as a yellow solid in 71% yield (97% purity by HPLC).

MS(ESI⁺): 389.8; MS(ESI⁻): 387.6.

### Example13

### [2-(cyclopentylamino)pyrimidin-4-yl](1-ethyl-1H-benzimidazol-2-yl)acetonitrile

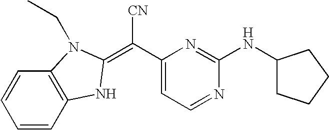

Following the general methods as outlined in Example 1 (Method C), (2-chloropyrimidin-4-yl)(1-ethyl-2,3-dihydro-(1H-benzimidazol-2-yl)acetonitrile (intermediate 7), and cyclopentylamine, the title compound was isolated, as a yellow solid in 75% yield (97% purity by HPLC).

MS (ESI+) 347.6, (ESI−) 345.8.

### Example 14

### 1,3-dihydro-2H-benzimidazol-2-ylidene(5-methyl-2-{[3-(2 oxopyrrolidin-1-yl)propyl]amino}pyrimidin-4-yl)acetonitrile

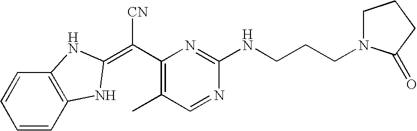

Following the general methods as outlined in Example 1 (Method C) (2-chloro-5-methylpyrimidin-4yl)(2,3-dihydro-(1H-benzimidazol-2-yl)acetonitrile (intermediate 8), and N-(3′-aminopropyl)-2-pyrrolidinone, the title compound was isolated, as a yellow solid in 76% yield (99% purity by HPLC).

MS(ESI⁺): 390.3; MS(ESI⁻): 388.6.

### Example 15

### (2Z)-(1-cyclobutyl-1,3-dihydro-2H-benzimidazol-2-ylidene)(2-{[3-(2-oxopyrrolidin-1-yl)propyl]amino}pyrimidin-4-yl)acetonitrile

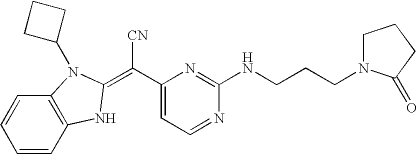

Following the general methods as outlined in Example 1 (Method C), starting from (2-chloropyrimidin-4-yl)(1-cyclobutyl-2,3-dihydro-1H-benzimidazol-2-yl)acetonitrile (intermediate 18), and N-(3′-aminopropyl)-2-pyrrolidinone, the title compound was isolated, as a yellow solid in 77% yield (98% purity by HPLC).

MS(ESI⁺): 430.6; MS(ESI⁻): 428.7.

### Example16

### (1-ethyl-1H-benzimidazol-2-yl){2-[(2-pyridin-3-ylethyl)amino]pyrimidin-4-yl}acetonitrile

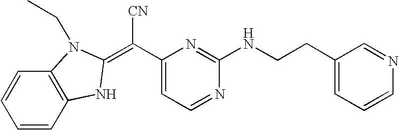

Following the general methods as outlined in Example 1 (Method D), (2-chloropyrimidin-4-yl)(1-ethyl-2,3-dihydro-(1H-benzimidazol-2-yl)acetonitrile (intermediate 7), and and 3-(2-aminoethyl)pyridine, the title compound was isolated, as a yellow solid in 70% yield (98% purity by HPLC).

MS(ESI³⁰ ): 384.4; MS(ESI⁻): 382.6.

### Example 17

### (2Z)-(1-ethyl-1,3-dihydro-2H-benzimidazol-2-ylidene)[2-(isobutylamino)-5-methylpyrimidin-4-yl]acetonitrile

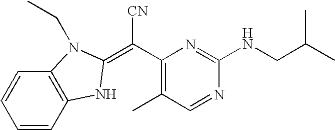

Following the general methods as outlined in Example 1 (Method D), starting from (2-chloro-5-methylpyrimidin-4yl)(1-ethyl-2,3-dihydro-1H-benzimidazol-2-yl)acetonitrile (intermediate 6), and isobutylamine, the title compound was isolated, as a yellow solid in 72% yield (99% purity by HPLC).

MS(ESI⁺): 349.6; MS(ESI⁻): 347.5.

### Example 18

### (2Z)-(1-ethyl-1,3-dihydro-2H-benzimidazol-2-ylidene)(2-{[2-(1H-imidazol-4-yl)ethyl]amino}pyrimidin-4-yl)acetonitrile

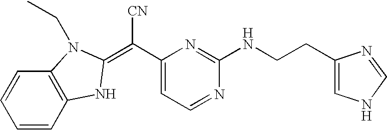

Following the general methods as outlined in Example 1 (Method C), (2-chloropyrimidin-4-yl)(1-ethyl-2,3-dihydro-1H-benzimidazol-2-yl)acetonitrile (intermediate 7), and histamine base, the title compound was isolated, as a yellow solid in 65% yield (95% purity by HPLC).

MS(ESI⁺): 373.3; MS(ESI⁻): 371.2.

### Example 19

### (2Z)-(1-ethyl-1,3-dihydro-2H-benzimidazol-2-ylidene)[2-(isobutylamino)pyrimidin-4-yl]acetonitrile

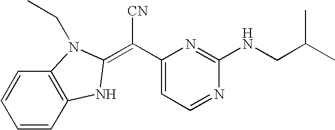

Following the general methods as outlined in Example 1 (Method C), (2-chloropyrimidin-4-yl)(1-ethyl-2,3-dihydro-1H-benzimidazol-2-yl)acetonitrile (intermediate 7), and isobutylamine, the title compound was isolated, as a yellow solid in 74% yield (99% purity by HPLC).

MS(ESI⁺): 335.4; MS(ESI⁻): 333.6.

### Example 20

### [2-(cyclopropylamino)pyrimidin-4-yl(1-ethyl-(1H-benzimidazol-2-yl]-cetonitrile

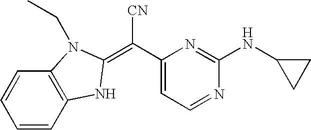

Following the general methods as outlined in Example 1 (Method C), (2-chloropyrimidin-4-yl)(1-ethyl-2,3-dihydro-1H-benzimidazol-2-yl)acetonitrile (intermediate 7), and cyclopropylamine, the title compound was isolated, as a yellow solid in 70% yield (98% purity by HPLC).

MS(ESI³⁰): 319.4; MS(ESI⁻): 317.3.

### Example21

### [2-({2-[6-(dimethylamino)pyridin-3-yl]ethyl}amino)pyrimidin-4-yl](1-ethyl-1H-benzimidazol-2-yl)acetonitrile

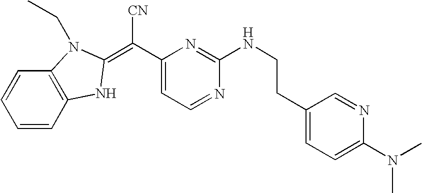

Following the general methods as outlined in Example 1 (Method C), (2-chloropyrimidin-4-yl)(1ethyl-2,3-dihydro-1H-benzimidazol-2-yl)acetonitrile (intermediate 7), and 2-(N,N-dimethylamino)-5-aminoethylpyridine, the title compound was isolated, as a yellow solid in 78% yield (96% purity by HPLC).

MS(ESI⁺): 427.5; MS(ESI⁻): 425.6.

### Example 22

### (1-ethyl-(1H-benzimidazol-2-yl)(2-{[2-(1H-1,2,4-triazol-1-yl)ethyl]amino}-pyrimidin-4yl)acetonitrile

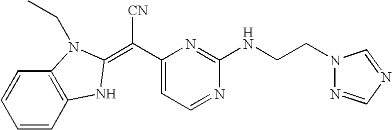

Following the general methods as outlined in Example 1 (Method D), (2-chloropyrimidin-4-yl)(1-ethyl-2,3-dihydro-(1H-benzimidazol-2-yl)acetonitrile (intermediate 7), and 2-(1H-1,2,4-triazole-1-YL)ethanamine hydrochloride, the title compound was isolated, as a yellow solid in 75% yield (99% purity by HPLC).

MS(ESI⁺): 374.6; MS(ESI⁻): 373.5.

### Example 23

### (2Z)-(1-ethyl-1,3-dihydro-2H-benzimidazol-2-ylidene(2-{[2-(1H-imidazol-4-yl)ethyl]amino}-5-methylpyrimidin-4-yl))acetonitrile

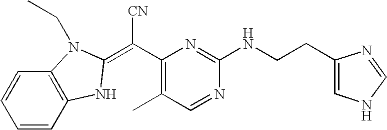

Following the general methods as outlined in Example 1 (Method D), starting from (2-chloro-5-methylpyrimidin-4-yl)(1-ethyl-2,3-dihydro-1H-benzimidazol-2-yl)acetonitrile (intermediate 6), and histamine base, the title compound was isolated, as a yellow solid in 69% yield (93% purity by HPLC).

MS(ESI⁺): 387.4; MS(ESI⁻): 385.6.

### Example 24

### [2-({2-[6-(dimethylamino)pyridin-3-yl]ethyl}amino)-5-methylpyrimidin-4-yl](1-ethyl-1H-benzimidazol-2-yl)acetonitrile

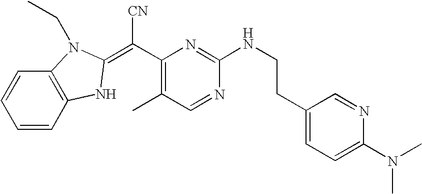

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and 2-N,N-dimethylamino)-5-aminoethylpyridine, the title compound was isolated, as a yellow solid in 68% yield (97% purity by HPLC).

MS(ESI⁺): 441.3; MS(ESI⁻): 439.5.

### Example 25

### (2Z)-[2-(cycloheptylamino)pyrimidin-4-yl](1-ethyl-1,3-dihydro-2H-benzimidazol-2-ylidene)acetonitrile

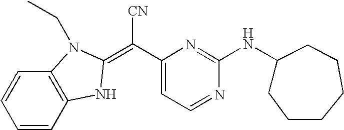

Following the general methods as outlined in Example 1 (Method C), (2-chloropyrimidin-4-yl)(1-ethyl-2,3-dihydro-1H-benzimidazol-2-yl)acetonitrile (intermediate 7), and cycloheptylamine, the title compound was isolated, as a yellow solid in 76% yield (98% purity by HPLC).

MS(ESI⁺): 375.7; MS(ESI⁻): 373.2.

### Example 26

### [2-(cyclopropylamino)-5-methylpyrimidin-4-yl](1-ethyl-1H-benzimidazol-2-yl)acetonitrile

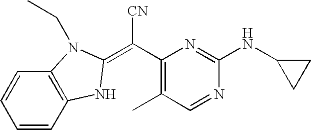

Following the general methods as outlined in Example 1 (Method D), stating from (2-chloro-5-methylpyrimidin-4-yl)(1-ethyl-2,3-dihydro-1H-benzimidazol-2-yl)acetonitrile (intermediate 6), and cyclopropylamine, the title compound was isolated, as a yellow solid in 75% yield (96% purity by HPLC).

MS(ESI⁺): 333.5; MS(ESI⁻): 331.3.

### Example 27

### (1-ethyl-1H-benzimidazol-2-yl){2-[(2-pyridin-2-ylethyl)amino]pyrimidin-4-yl}acetonitrile

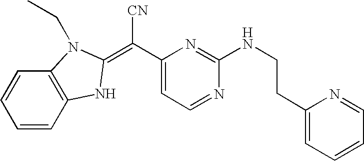

Following the general methods as outlined in Example 1 (Method C), (2-chloropyrimidin-4-yl)(1-ethyl-2,3-dihydro-(1H-benzimidazol-2-yl)acetonitrile(intermediate7), and 2-(2-aminoethyl)pyridine, the title compound was isolated, as a yellow solid in 80% yield (94% purity by HPLC).

MS(ESI⁺): 384.8; MS(ESI⁻): 382.2.

### Example 28

### [2-(cyclopentylamino)-5-methylpyrimidin-4yl](1,3-dihydro-2H-benzimidazol-2-ylidene)acetonitrile

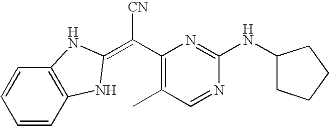

Following the general methods as outlined in Example 1 (Method C), (2-chloro-5-methylpyrimidin-4-yl)(2,3-dihydro-1H-benzimidazol-2-yl)acetonitrile (intermediate 8), and cyclopentylamine, the title compound was isolated, as a yellow solid in 80% yield (77% purity by HPLC).

MS(ESI⁺): 333.3; MS(ESI⁻): 331.2.

### Example 29

### [2-(cyclohexylamino)pyrimidin-4-yl](1-ethyl-1H-benzimidazol-2-yl)acetonitrile

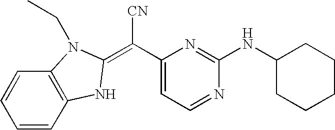

Following the general methods as outlined in Example 1 (Method C), (2-chloropyrimidin-4-yl)(1-ethyl-2,3-dihydro-(1H-benzimidazol-2-yl)acetonitrile (intermediate 7), and cyclohexylamine, the title compound was isolated, as a yellow solid in 70% yield (97% purity by HPLC).

MS(ESI⁺): 361.2; MS(ESI⁻): 359.4.

### Example 30

### (2Z)-(1-ethyl-1,3-dihydro-2H-benzimidazol-2-ylidene)(2-{[2-(1H-indol-3-yl)ethyl]amino}-5-methylpyrimidin4-yl)acetonitrile

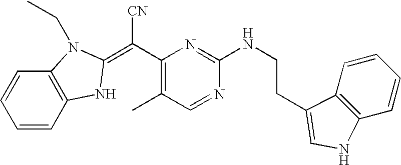

Following the general methods as outlined in Example 1 (Method C), starting from (2-chloro-5-methylpyrimidin-4yl)(1-ethyl-2,3-dihydro-1H-benzimidazol-2-yl)acetonitrile (intermediate 6), and tryptamine, the title compound was isolated, as a yellow solid in 77% yield (98% purity by HPLC).

MS(ESI⁺): 436.5; MS(ESI⁻): 434.6.

### Example 31

### (1-ethyl-(1H-benzimidazol-2-yl){5-methyl-2-[(2-pyridin-2-ylethyl)amino]pyrimidin-4-yl}acetonitrile

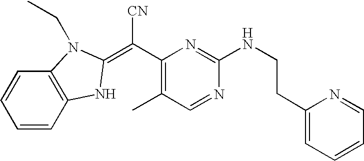

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and 2-(2-aminoethyl)pyridine), the title compound was isolated, as a yellow solid in 77% yield (98% purity by HPLC).

MS(ESI⁺): 398.6; MS(ESI⁻): 396.4.

### Example 32

### {2-[(2-ethoxyethyl)amino]pyrimidin-4-yl}(1-ethyl-1H-benzimidazol-2-yl)acetonitrile

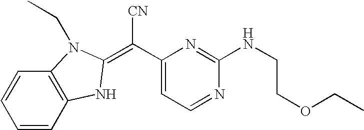

Following the general methods as outlined in Example 1 (Method D), (2-chloropyrimidin-4-yl)(1ethyl-2,3-dihydro-(1H-benzimidazol-2-yl)acetonitrile (intermediate 7), and 2-ethoxyethylamine, the title compound was isolated, as a yellow solid in 78% yield (92% purity by HPLC).

MS(ESI⁺): 351.7; MS(ESI⁻): 349.6.

### Example 33

### (1-ethyl-1H-benzimidazol-2-yl){5-methyl-2-[(1-methylbutyl)amino]pyrimidin-4-yl}acetonitrile

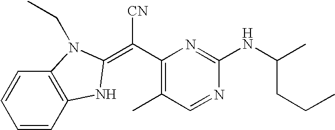

Following the general methods as outlined in Example 1 (Method D), starting from (2-chloro-5-methylpyrimidin-4-yl)(1-ethyl-2,3-dihydro-1H-benzimidazol-2-yl)acetonitrile (intermediate 6), and (+/−)-2-aminopentane, the title compound was isolated, as a yellow solid in 70% yield (92% purity by HPLC).

MS(ESI⁺): 363.5; MS(ESI⁻): 361.8.

### Example 34

### (1-ethyl-1H-benzimidazol-2-yl)[2-(methylamino)pyrimidin-4yl]acetonitrile

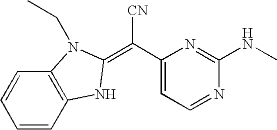

Following the general methods as outlined in Example 1 (Method D), (2-chloropyrimidin-4-yl)(1-ethyl-2,3-dihydro-(1H-benzimidazol-2-yl)acetonitrile (intermediate 7), and methylamine, the title compound was isolated, as a yellow solid in 78% yield (98% purity by HPLC).

MS(ESI⁺): 293.4; MS(ESI⁻): 291.6.

### Example 35

### (1-ethyl-1H-benzimidazol-2-yl)(5-methyl-2-{[2-(1H-pyrazol-1-yl)ethyl]amino}pyrimidin-4-yl)acetonitrile

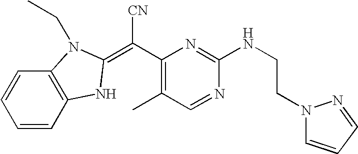

Following the general methods as outlined in Example 1 (Method D), starting from (2-chloro-5-methylpyrimidin-4yl)(1-ethyl-2,3-dihydro-1H-benzimidazol-2-yl)acetonitrile (intermediate 6), and 1-(2′-aminoethyl)pyrazole), the title compound was isolated, as a yellow solid in 74% yield (97% purity by HPLC).

MS(ESI⁺): 387.6; MS(ESI⁻): 385.2.

### Example 36

### (1H-benzimidazol-2-yl{5-methyl-2-[(2-pyridin-3-ylethyl)amino]pyrimidin-4-yl}acetonitrile

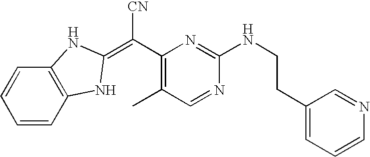

Following the general methods as outlined in Example 1 (Method C), (2-chloro-5-methylpyrimidin-4-yl)(2,3-dihydro-1H-benzimidazol-2-yl)acetonitrile (intermediate 8), and 3-(2-aminoethyl)pyridine, the title compound was isolated, as a yellow solid in 68% yield (88% purity by HPLC).

MS(ESI⁺): 370.3; MS(ESI⁻): 368.8.

### Example 37

### (2Z)-(1-ethyl)-1,3-dihydro-2H-benzimidazol-2-ylidene)(2-{[2-(1H-imidazol-1-yl)ethyl]amino}-5-methylpyrimidin-4-yl)acetonitrile

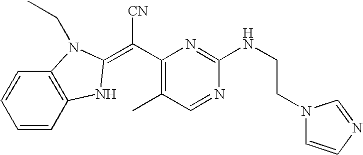

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and 2-(imidazol-1-yl)-ethylamine, the title compound was isolated, as a yellow solid in 60% yield (97% purity by HPLC).

MS(ESI⁺): 387.2; MS(ESI⁻): 385.4.

### Example 38

### 1H-benzimidazol-2-yl{2-[(2-pyridin-3-ylethyl)amino]pyrimidin-4-yl}acetonitrile

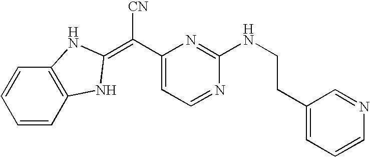

Following the general methods as outlined in Example 1 (Method C), (2-chloropyrimidin-4-yl)(2,3-dihydro-(1H-benzimidazol-2-yl)acetonitrile (intermediate 9), and 3-(2-aminoethyl)pyridine, the title compound was isolated, as a yellow solid in 73% yield (98% purity by HPLC).

MS(ESI⁺): 356.2; MS(ESI⁻): 354.3.

### Example 39

### (1-ethyl-1H-benzimidazol-2-yl){2-[(1-methylbutyl)amino]pyrimidin-4-yl}acetonitrile

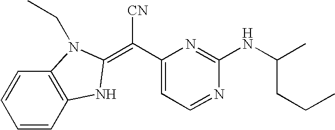

Following the general methods as outlined in Example 1 (Method D), (2-chloropyrimidin-4-yl)(1-ethyl-2,3-dihydro-1H-benzimidazol-2-yl)acetonitrile (intermediate 7), and (+/−)-2-aminopentane, the title compound was isolated, as a yellow solid in 74% yield (97% purity by HPLC).

MS(ESI⁺): 349.4; MS(ESI^{-):} 347.2.

### Example 40

### {2-[(cyclohexylmethyl)amino]-5-methylpyrimidin-4-yl}(1-ethyl-1H-benzimidazol-2-yl)acetonitrile

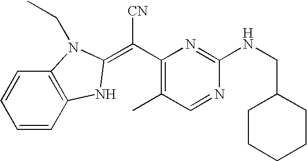

Following the general methods as outlined in Example 1 (Method C), starting from (2-chloro-5-methylpyrimidin-4-yl)(1-ethyl-2,3-dihydro-1H-benzimidazol-2-yl)acetonitrile (intermediate 6), and (aminomethyl)cyclohexane, the title compound was isolated, as a yellow solid in 77% yield (94% purity by HPLC).

MS(ESI⁺): 389.5; MS(ESI⁻): 387.6.

### Example 41

### 1H-benzimidazol-2-yl[2-(cyclopentylamino)pyrimidin-4-yl]acetonitrile

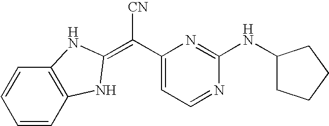

Following the general methods as outlined in Example 1 (Method C), (2-chloropyrimidin-4-yl)(2,3-dihydro-1H-benzimidazol-2-yl)acetonitrile (intermediate 9), and cyclopentyamine, the title compound was isolated, as a yellow solid in 74% yield (96% purity by HPLC).

MS(ESI⁺): 319.4; MS(ESI⁻): 317.3.

### Example 42

### (1-ethyl-1H-benzimidazol-2-yl){6-methyl-2-[(2-pyridin-3-ylethyl)amino]pyrimidin-4-yl}acetonitrile

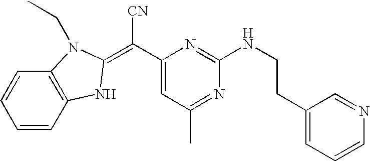

Following the general methods as outlined in Example 1 (Method D), (2-chloro-6-methylpyrimidin-4-yl)(1-ethyl-2,3-dihydro-1H-benzimidazol-2-yl)acetonitrile (intermediate 10), and 3-(2-aminoethyl)pyridine, the title compound was isolated, as a yellow solid in 72% yield (97% purity by HPLC).

MS(ESI⁺): 398.5; MS(ESI⁻): 396.4.

### Example 43

### 1H-benzimidazol-2-yl[2-(cyclopropylamino)pyrimidin-4-yl]acetonitrile

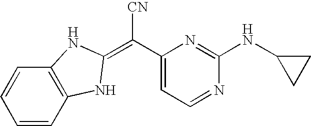

Following the general methods as outlined in Example 1 (Method D), (2-chloropyrimidin-4-yl)(2,3-dihydro-1H-benzimidazol-2-yl)acetonitrile (intermediate 9), and cyclopropylamine, the title compound was isolated, as a yellow solid in 79% yield (97% purity by HPLC).

MS(ESI⁺): 291.4; MS(ESI⁻): 289.4.

### Example 44

### [2-(cyclopentylamino)-6-methylpyrimidin-4-yl](1-ethyl-1H-benzimidazol-2-yl)acetonitrile

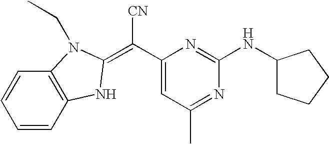

Following the general methods as outlined in Example 1 (Method D), (2-chloro-6-methylpyrimidin4-yl)(1-ethyl-2,3-dihydro-(1H-benzimidazol-2-yl)acetonitrile (intermediate 10), and cyclopentylamine, the title compound was isolated, as a yellow solid in 72% yield (98% purity by HPLC).

MS(ESI⁺): 361.6; MS(ESI⁻): 359.5.

### Example 45

### {2-[(cyclohexylmethyl)amino]pyrimidin-4-yl}(1-ethyl-(1H-benzimidazol-2-yl)acetonitrile

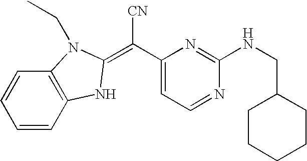

Following the general methods as outlined in Example 1 (Method D), (2-chloropyrimidin-4yl)(1-ethyl-2,3-dihydro-(1H-benzimidazol-2-yl)acetonitrile (intermediate 7), and (aminomethyl)cyclohexane, the title compound was isolated, as a yellow solid in 72% yield (98% purity by HPLC).

MS(ESI⁺): 375.2; MS(ESI⁻): 373.2.

### Example 46

### (1-ethyl-(1H-benzimidazol-2-yl){6-[(2-pyridin-3-ylethyl)amino]pyrimidin-4-yl}acetonitrile

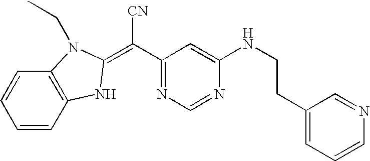

Following the general methods as outlined in Example 1 (Method D), starting from (6-chloropyrimidin-4-yl)(1-ethyl-2,3-dihydro-(1H-benzimidazol-2-yl)acetonitrile (intermediate 11), and 3-(2-aminoethyl)pyridine, the title compound was isolated, as a yellow solid in 72% yield (88% purity by HPLC).

MS(ESI⁺): 384.6; MS(ESI⁻): 382.4.

### Example 47

### (1-ethyl-(1H-benzimidazol-2-yl){2-[(3-pyrrolidin-1-ylpropyl)amino]-pyrimidin-4-yl}acetonitrile

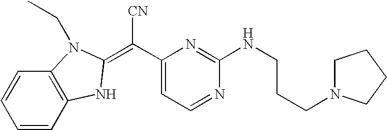

Following the general methods as outlined in Example 1 (Method D), (2-chloropyrimidin-4yl)(1-ethyl-2,3-dihydro-(1H-benzimidazol-2-yl)acetonitrile (intermediate 7), and 1-amino-3-(N-piperidino)propane, the title compound was isolated, as a yellow solid in 74% yield (94% purity by HPLC).

MS(ESI⁺): 390.4; MS(ESI⁻): 388.2.

### Example 48

### (1-ethyl-1H-benzimidazol-2-yl)[2-(4-ethylpiperazin-1-yl-5-methylpyrimidin-4-yl]acetonitrile

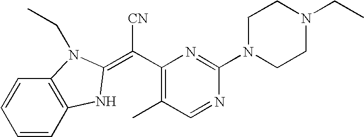

Following the general methods as outlined in Example 1 (Method D), starting from (2-chloro-5-methylpyrimidin-4-yl)(1-ethyl-2,3-dihydro-1H-benzimidazol-2-yl)acetonitrile (intermediate 6), and 1-ethylpiperazine, the title compound was isolated, as a yellow solid in 76% yield (99% purity by HPLC).

MS(ESI⁺): 390.5; MS(ESI⁻): 388.4.

### Example 49

### (1-ethyl-1H-benzimidazol-2-yl){2-[(2-furylmethyl)amino]-5-methylpyrimidin-4-yl}acetonitrile

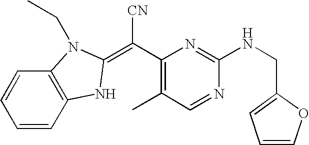

Following the general methods as outlined in Example 1 (Method D), starting from (2-chloro-5-methylpyrimidin-4-yl)(1-ethyl-2,3-dihydro-1H-benzimidazol-2-yl)acetonitrile (intermediate 6), and furfurylamine, the title compound was isolated, as a yellow solid in 71% yield (92% purity by HPLC).

MS(ESI⁺): 373.3; ESI⁻): 371.5.

### Example 50

### (2Z)-(1-ethyl-1,3-dihydro-2H-benzimidazol-2-ylidene){5-methyl-2-[(1-methylpiperidin-4yl)amino]pyrimidin-4-yl}acetonitrile

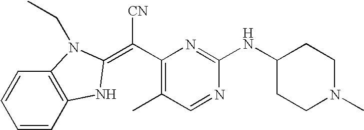

Following the general methods as outlined in Example 1 (Method D), starting from (2-chloro-5-methylpyrimidin-4yl)(1-ethyl-2,3-dihydro-1H-benzimidazol-2-yl)acetonitrile (intermediate 6), and 4-amino-1-methyl-piperidine, the title compound was isolated, as a yellow solid in 79% yield (98% purity by HPLC).

MS(ESI⁺): 390.2; MS(ESI⁻): 388.3.

### Example 51

### (2Z)-[2-(cyclohexylamino)-5-methlypyrimidin-4-yl](1-ethyl-1,3-dihydro-2H-benzimidazol-2-ylidene)acetonitrile

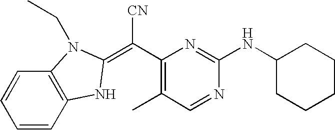

Following the general methods as outlined in Example 1 (Method C), starting from (2-chloro-5-methylpyrimidin-yl)(1-ethyl-2,3-dihydro-1H-benzimidazol-2-yl)acetonitrile (intermediate 6), and cyclohexylamine, the title compound was isolated, as a yellow solid in 74% yield (97% purity by HPLC).

MS(ESI⁺): 375.8; MS(ESI⁻): 373.2.

### Example 52

### (2Z)-[2-(ethylamino)-5-methylpyrimidin-4-yl](1-ethyl-1,3-dihydro-2H-benzimidazol-2-ylidene)acetonitrile

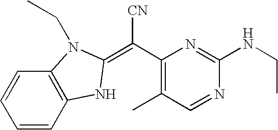

Following the general methods as outlined in Example 1 (Method D), starting from (2-chloro-5-methylpyrimidin-4-yl)(1-ethyl-2,3-dihydro-(1H-benzimidazol-2-yl)acetonitrile (intermediate 6), and ethylamine, the title compound was isolated, as a yellow solid in 76% yield (99% purity by HPLC).

MS(ESI⁺): 321.2; MS(ESI⁻): 319.3.

### Example 53

### [2-(cyclopentylamino)-5-methylpyrimidin-4-yl](1,3-diethyl-1,3-dihydro-2H-benzimidazol-2-ylidene)acetonitrile

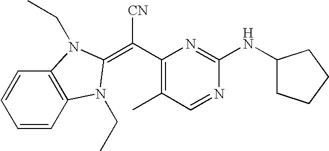

Following the general methods as outlined in Example 1 (Method D), starting from (2-chloro-5-methylpyrimidin-4-yl)(1,3-diethyl-2,3-dihydro-1H-benzimidazol-2-yl)acetonitrile (intermediate 15), and cyclopentylamine, the title compound was isolated, as a yellow solid in 80% yield (97% purity by HPLC).

MS(ESI⁺): 389.2; MS(ESI⁻): 387.3.

### Example 54

### (2Z)-(1-ethyl-1,3-dihydro-2H-benzimidazol-2-ylidene)[5-methyl-2-(piperidin-4-ylamino)pyrimidin-4yl]acetonitrile

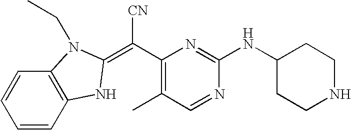

Following the general methods as outlined in Example 1 (Method D), starting from (2-chloro-5-methylpyrimidin-4yl)(1-ethyl-2,3-dihydro-1H-benzimidazol-2-yl)acetonitrile (intermediate 6), and 4-amino-1-piperidine, the title compound was isolated, as a yellow solid in 70% yield (97% purity by HPLC).

MS(ESI⁺): 376.2; MS(ESI⁻): 374.3.

### Example 55

### (2Z)-(1-ethyl-1,3-dihydro-2H-benzimidazol-2-ylidene){5-methyl-2-[(2piperidin-1-ylethyl)amino]pyrimidin-4-yl}acetonitrile

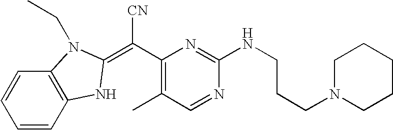

Following the general methods as outlined in Example 1 (Method D), starting from (2-chloro-5-methylpyrimidin-4-yl)(1-ethyl-2,3-dihydro-(1H-benzimidazol-2-yl)acetonitrile (intermediate 6), and 1-amino-3-(N-piperidino)propane, the title compound was isolated, as a yellow solid in 70% yield (97% purity by HPLC).

MS(ESI⁺): 418.6; MS(ESI⁻): 416.2.

### Example 56

### General Procedure for the Solution-Phase Synthesis of benzimidazoles acetonitriles Derivatives of General Formula I, with G and L as above Defined (Schemes 10); tert-butyl (4S)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate

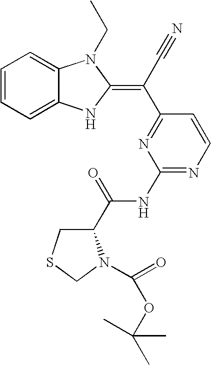

To a solution of (2Z)-(2-amino-pyrimidin-4-yl)(1-ethyl-1,3-dihydro-2H-benzimidazol-2-ylidene)acetonitrile (intermediate 20) (300 mg, 1.08 mmol, 1 eq) in 8 mL of DCM:THF (6:2)) were added Boc-D-thiazolidine-4-carboxylic acid 8377 mg, 1.62 mmol, 1.5 eq) followed by the Mukaiyama reagent (826 mg, 3.23 mmol, 3 eq) and DIEA (371 μL, 2.16 mmol, 2 eq). The reaction mixture was stirred at room temperature for 6 days. The reaction mixture was diluted with DCM (50 mL), washed with NH4Cl, NaHCO3 and brine and dried over MgSO₄. The solvent was removed by evaporation and the residue purified by FC using a gradient AcOEt:CycloH (4:6) to neat AcOEt then to AcOEt:MeOH (7:3) for 1 hour. The expected product tert-butyl (4S)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate was isolated as a yellow solid (292 mg, 55% yield, 99% HPLC purity

MS (ESI+), 494.5, ESI−), 492.3.

### Example 57

### (4S)-N-{4-[(Z)-cyano(1-ethyl-1,3-dihydro-2H-benzimidazol-2-ylidene)methyl]-pyrimidin-2-yl}-1,3-thiazolidine-4-carboxamide

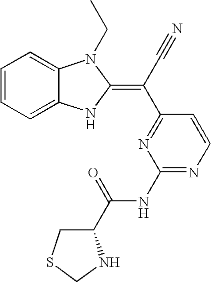

To a solution of tert-butyl (4S)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate (292 mg, 0.591 mmol) was added at zero degree a solution of 10% TFA in DCM (10 mL) and the reaction mixture was stirred for 1 h. The solvent was evaporated and the expected product (4S)-N-{4-[(Z)-cyano(1-ethyl-1,3-dihydro-2H-benzimidazol-2-ylidene)methyl]-pyrimidin-2-yl}-1,3-thiazolidine-4-carboxamide was isolated as a yellow solid (252 mg, 98% yield, 99% HPLC purity)

MS(ESI⁺): 394.5; MS(ESI⁻): 392.2.

### Example 58

### N-{4-[(Z)-cyano(1-ethyl-1,3-dihydro-2H-benzimidazol-2-ylidene)methyl]-5-methylpyrimidin-2-yl}-4-(4-methylpiperazin-1-yl)-4-oxobutanamide

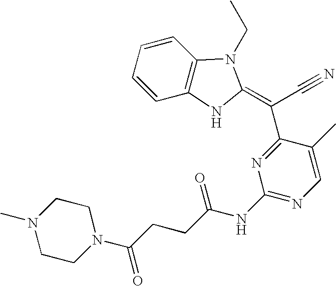

Following the general method as outlined in Example 56, starting from (2Z)-(2-amino-5-methylpyrimidin-4yl)(1-ethyl-1,3-dihydro-2H-benzimidazol-2-ylidene)acetonitrile (Intermediate 19), and 4-(4-methyl-piperazin-1-yl)-4-oxo-butyric acid, the title compound was isolated, as a yellow solid in 65% yield (99% purity by HPLC).

MS(ESI⁺): 475.6; MS(ESI⁻): 473.6.

### Example 59

### N-{4-[(Z)-cyano(1-ethyl-1,3-dihydro-2H-benzimidazol-2-ylidene)methyl]-pyrimidin-2-yl}-4-(4-methylpiperazin-1-yl)-4-oxobutanamide

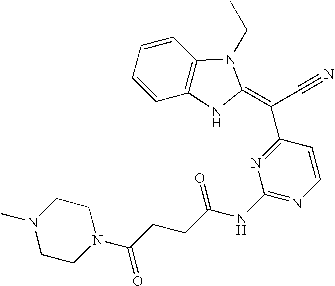

Following the general method as outlined in Example 56, starting (2Z)-(2-amino-pyrimidin-4-yl)(1-ethyl-1,3-dihydro-2H-benzimidazol-2-ylidene)acetonitrile (intermediate 20), and 4-(4-methyl-piperazin-1-yl)-4-oxo-butyric acid, the title compound was isolated, as a yellow solid in 65% yield (89% purity by HPLC).

MS(ESI⁺): 461.6; MS(ESI⁻): 459.2.

### Example 60

### (1R,5R,7R)-N-{4-[(Z)-cyano(1-ethyl-1,3-dihydro-2H-benzimidazol-2-ylidene)methyl]-5-methylpyrimidin-2-yl}-6,8-dioxa-3-azabicyclo3.2.1]octane-7-carboxamide

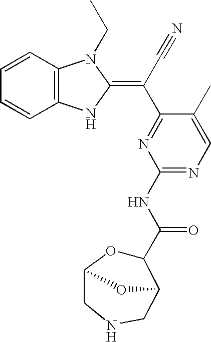

Following the general methods as outlined in Examples 56 and 57, starting from (2Z)-(2-amino-5-methylpyrimidin-4-yl)(1-ethyl-1,3-dihydro-2H-benzimidazol-2-ylidene)-acetonitrile (Intermediate 19), and Boc-7-endo-BTG-OH, the title compound was isolated, as a yellow solid in 71% yield (98% purity by HPLC).

MS(ESI⁺): 434.5; MS(ESI⁻): 432.3.

### Example 61

### (1S,5S,7S)-N-{4-[(Z)-cyano(1-ethyl-1,3-dihydro-2H-benzimidazol-2-ylidene)methyl]-5-methylpyrimidin-2-yl}-6,8-dioxa-3-azabicyclo[3.2.1]octane-7-carboxamide

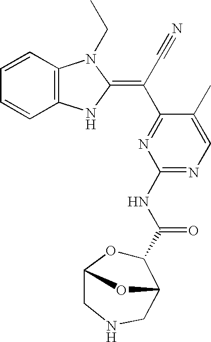

Following the general methods as outlined in Example 56 and 57, starting from (2Z)-(2-amino-5-methylpyrimidin-4-yl)(1-ethyl-1,3-dihydro-2H-benzimidazol-2-ylidene)-acetonitrile (Intermediate 19), and Boc-7-endo-BTG-OH, the title compound was isolated, as a yellow solid in 72% yield (97% purity by HPLC).

MS(ESI⁺): 434.5; MS(ESI⁻): 433.4.

### Example 62

### 4-tert-butyl 1-(9H-fluoren-9-ylmethyl)2-[({4-[(Z)-cyano(1-ethyl-1,3-dihydro-2H-benzimidazol-2-ylidene)methyl]-5-methylpyrimidin-2-yl}amino)carbonyl]piperazine-1,4-dicarboxylate

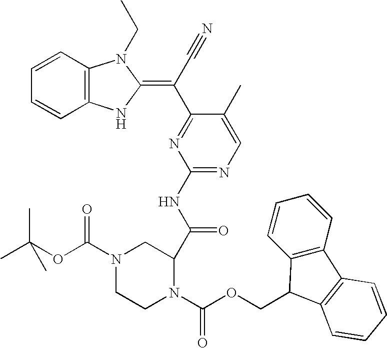

Following the general method as outlined in Example 56, starting from (2Z)-(2-amino-5-methylpyrimidin-4-yl)(1-ethyl-1,3-dihydro-2H-benzimidazol-2-ylidene)acetonitrile (intermediate 19), and 1-fmoc-4-boc-piperazine-2-carbocxylic acid, the title compound was isolated, as a yellow solid in 70% yield (97% purity by HPLC).

MS(ESI⁺): 727.9; MS(ESI⁻): 725.6.

### Example 63

### N-{4-[(Z)-cyano(1-ethyl-1,3-dihydro-2H-benzimidazol-2-ylidene)methyl]-5-methylpyrimidin-2-yl}piperazine-2-carboxamide

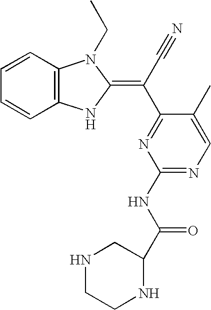

Following the general method as outlined in Example 57, starting 4-tert-butyl 1-(9H-fluoren-9-ylmethyl)2-[({4-[(Z)-cyano(1-ethyl-1,3-dihydro-2H-benzimidazol-2-ylidene)methyl]-5-methylpyrimidin-2-yl}amino)carbonyl]piperazine-1,4-dicarboxylate (Example 63), the title compound was isolated, as a yellow solid in 90% yield (91% purity by HPLC).

MS(ESI⁺): 404.6; MS(ESI⁻): 402.2.

### Example 64

### tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-5-oxopyrrolidine-1-carboxylate

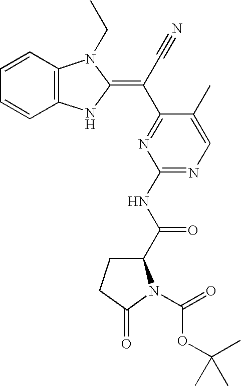

Following the general method as outlined in Example 56, starting from (2Z)-(2-amino-5-methylpyrimidin-4yl)(1-ethyl-1,3-dihydro-2H-benzimidazol-2-ylidene)acetonitrile (Intermediate 19), and Boc-pyroglutamic acid, the title compound was isolated, as a yellow solid in 72% yield (98% purity by HPLC).

MS(ESI⁺): 504.3; MS(ESI⁻): 502.2.

### Example 65

### N-{4-[(Z)-cyano(1-ethyl-1,3-dihydro-2H-benzimidazol-2-ylidene)methyl]-5-methylpyrimidin-2-yl}-5-oxo-L-prolinamide

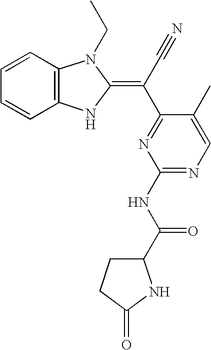

Following the general method as outlined in Example 57, starting from: tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-5-oxopyrrolidine-1-carboxylate (Example 65), the title compound was isolated, as a yellow solid in 96% yield (97% purity by HPLC).

MS(ESI⁺): 404.6; MS(ESI⁻): 402.2.

### Example 66

### tert-butyl (4R)-4-9 ({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate

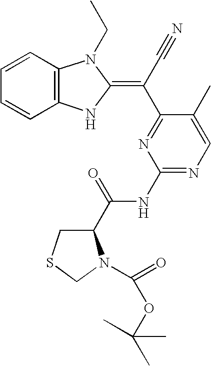

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and (S)-boc-5,5-dimethyl-1,3-thiazolidine-4-carboxylic acid, the title compound was isolated, as a yellow solid in 80% yield (98% purity by HPLC).

MS(ESI⁺): 508.9; MS(ESI⁻): 506.5.

### Example 67

### (4R)-N-{4-[(Z)-cyano(1-ethyl-1,3-dihydro-2H-benzimidazol-2-ylidene)methyl]-5-methlypyrimidin-2-yl}-1,3-thiazolidine-4-carboxamide

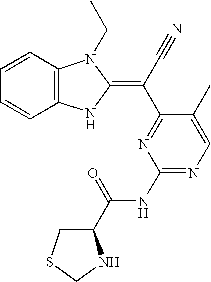

Following the general method as outlined in Example 57, starting tert-butyl (4R)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate (Example 66), the title compound was isolated, as a yellow solid in 94% yield (97% purity by HPLC).

MS(ESI⁺): 408.6; MS(ESI⁻): 406.2.

### Example 68

### (1S,4S,5S,7R)-N-{4-[(Z)-cyano(1-ethyl-1,3-dihydro-2H-benzimidazol-2-ylidene)methyl]-5-methylpyrimidin-2-yl}-4-methyl-6,8-dioxa-3-azabicyclo[3.2.1]octane-7-carboxamide

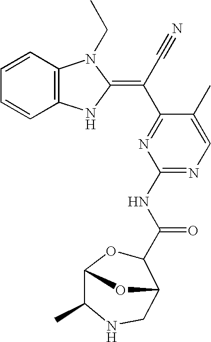

Following the genera methods as outlined in Examples 56 and 57, starting from (2Z)-(2-amino-5-methylpyrimidin-4-yl)(1-ethyl-1,3-dihydro-2H-benzimidazol-2-ylidene)-acetonitrile (Intermediate 19), and Boc-BTA-OH, the title compound was isolated, as a yellow solid in 80% yield (93% purity by HPLC).

MS(ESI⁺): 448.7; MS(ESI⁻): 446.3.

### Example 69

### N-{4-[(Z)-cyano(1-ethyl-1,3-dihydro-2H-benzimidazol-2-ylidene)methyl]-5-methylpyrimidin-2-yl}-4-(dimethylamino)butanamide

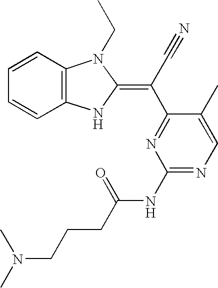

Following the general method as outlined in Example 56, starting from (2Z)-(2-amino-5-methylpyrimidin-4yl)(1-ethyl-1,3 dihydro-2H-benzimidazol-2-ylidene)acetonitrile (Intermediate 19), and 4-(dimethylamino)butyric acid hydrochloride, the title compound was isolated, as a yellow solid in 50% yield (97% purity by HPLC).

MS(ESI⁺): 406.6; MS(ESI⁻): 404.2.

### Example 70

### N˜1˜˜{4-[(Z)-cyano(1-ethyl-1,3-dihydro-2H-benzimidazol-2-ylidene)methyl]-5-methylpyrimidin-2-yl}-N˜3˜,N˜3˜-dimethyl-beta-alaninamide

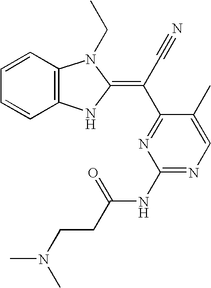

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 3-(dimethylamino)propanoic acid, the title compound was isolated, as a yellow solid in 80% yield (94% purity by HPLC).

MS(ESI⁺): 392.5; MS(ESI⁻): 390.6.

### Example 71

### N-{4-[(Z)-cyano(1-ethyl-1,3-dihydro-2H-benzimidazol-2-ylidene)methyl]-5-methylpyrimidin-2-yl}-4-(4-methylpiperazin-1-yl)butanamide

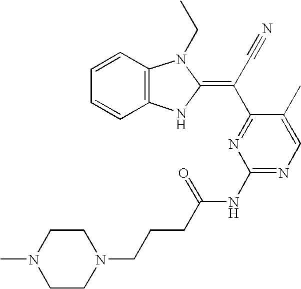

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and (4methylpiperazin-1-yl)butanoic acid, the title compound was isolated, as a yellow solid in 52% yield (96% purity by HPLC).

MS(ESI⁺): 461.7; MS(ESI⁻): 459.2.

### Example 72

### N-{4-[(Z)-cyano(1ethyl-1,3-dihydro-2H-benzimidazol-2-ylidene)methyl]-5-methylpyrimidin-2-yl}cyclopentanecarboxamide

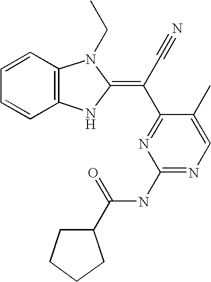

Following the general method as outlined in Example 56, starting from (2Z)-(2-amino-5-methylpyrimidin-4-yl)(1-ethyl-1,3 dihydro-2H-benzimidazol-2-ylidene)acetonitrile (Intermediate 19), and cyclopentanopic acid, the title compound was isolated, as a yellow solid in 65% yield (97% purity by HPLC).

MS(ESI⁺): 389.5; MS(ESI⁻): 387.6.

### Example 73

### tert-butyl[4-({4-[(Z)-cyano(1-ethyl-1,3-dihydro-2H-benzimidazol-2-ylidene)methyl]-5-methylpyrimidin-2-yl}amino)-4oxobutyl]carbamate

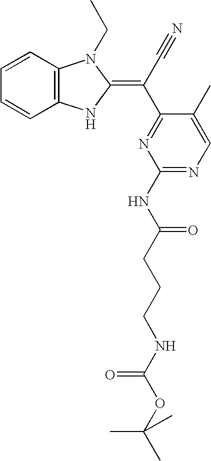

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 4-(boc-amino)butyric acid, the title compound was isolated, as a yellow solid in 48% yield (95% purity by HPLC).

MS(ESI⁺): 478.6; MS(ESI⁻): 476.5.

### Example 74

### 4-amino-N-{4-[(Z)-cyano(1-ethyl-1,3-dihydro-2H-benzimidazol-2-ylidene)methyl]-5-methylpyrimidin-2-yl}butanamide

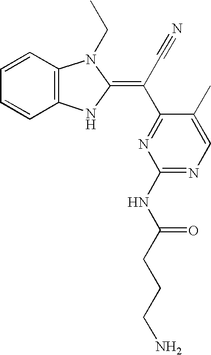

Following the general methods as outlined in Examples 56 and 57, starting from tert-butyl[4-({4-[(Z)-cyano(1-ethyl-1,3-dihydro-2H-benzimidazol-2-ylidene)methyl]-5-methylpyrimidin-2-yl}amino)-4-oxobutyl]carbamate (Example 73), the title compound was isolated, as a yellow solid in 90% yield (96% purity by HPLC).

MS(ESI⁺): 378.6; MS(ESI⁻): 376.7.

### Example 75

### tert-butyl 4-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate

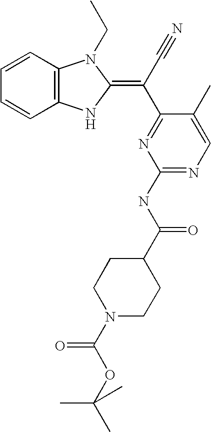

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 1-(tert-butoxycarbonyl)isonipecotic acid, the title compound was isolated, as a yellow solid in 84% yield (98% purity by HPLC).

MS(ESI⁺): 504.6; MS(ESI⁻): 502.3.

### Example 76

### N-{4-[(Z)-cyano(1-ethyl-1,3-dihydro-2H-benzimidazol-2-ylidene)methyl]-5-methylpyrimidin-2-yl}piperidine-4-carboxamide

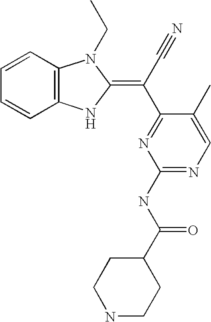

Following the general methods as outlined in Examples 56 and 57, starting from tert-butyl 4-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate (Example 75), the title compound was isolated, as a yellow solid in 95% yield (97% purity by HPLC).

MS(ESI⁺): 404.6; MS(ESI⁻): 402.8.

### Example 77

### N-{4-[(Z)-cyano(1-ethyl-1,3-dihydro-2H-benzimidazol-2-ylidene)methyl]-5-methylpyrimidin-2-yl}-1-methylpiperidine-4-carboxamide

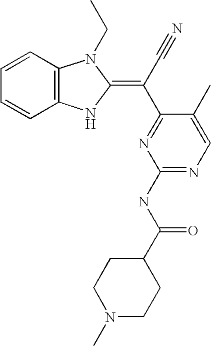

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 1-methyl-piperidine-4-carboxylic acid HCl, the title compound was isolated, as a yellow solid in 82% yield (92% purity by HPLC).

MS(ESI⁺): 418.8; MS(ESI⁻): 416.5.

### Example 78

### 1-acetyl-N-{4-[(Z)-cyano(1-ethyl-1,3-dihydro-2H-benzimidazol-2-ylidene)methyl]-5-methylpyrimidin-2-yl}piperidine-4-carboxamide

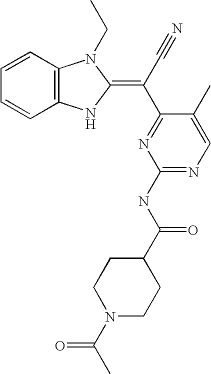

Following the general method as outlined in Example 56, starting from (2Z)-(2-amino-5-methylpyrimidin-4-yl)(1-ethyl-1,3-dihydro-2H -benzimidazol-2-ylidene)acetonitrile (Intermediate 19), and 1-acetyl-4-piperidine carboxylic acid, the title compound was isolated, as a yellow solid in 82% yield (98% purity by HPLC).

MS(ESI⁺): 446.5; MS(ESI⁻): 444.9.

### Example 79

### N-{4-[(Z)-cyano(1-ethyl-1,3-dihydro-2H-benzimidazol-2-ylidene)methyl]-5-methylpyrimidin-2-yl}-2-(2-methoxyphenyl)acetamide

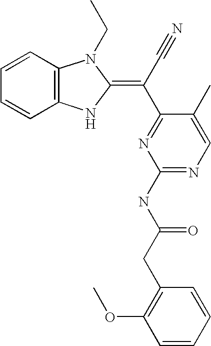

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 2-methoxy-phenylacetic acid, the title compound was isolated, as a yellow solid in 60% yield (95% purity by HPLC).

MS(ESI⁺): 441.8; MS(ESI⁻): 439.4.

### Example 80

### tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-2,5-dihydro-1H-pyrrole-1-carboxylate

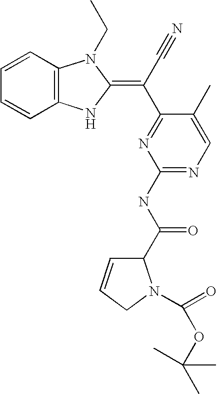

Following the general method as outlined in Example 56, starting from (2Z)-(2-amino-5-methylpyrimidin-4yl)(1-ethyl-1,3-dihydro-2H-benzimidazol-2-ylidene)acetonitrile (Intermediate 19), and boc-3,4-dehydro-L-proline, the title compound was isolated, as a yellow solid in 65% yield (99% purity by HPLC).

MS(ESI⁺): 488.6; MS(ESI⁻): 486.6.

### Example 81

### (2S)-N-{(4-[(Z)-cyano(1-ethyl-1,3-dihydro-2H-benzimidazol-2-ylidene)methyl]-5-methylpyrimidin-2-yl}-2,5-dihydro-1H-pyrrole-2-carboxamide

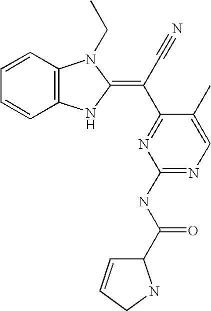

Following the general method as outlined in Example 56, starting from tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-2,5-dihydro-1H-pyrrole-1-carboxylate (Example 80), the title compound was isolated, as a yellow solid in 95% yield (96% purity by HPLC).

MS(ESI⁺): 388.5; MS(ESI⁻): 386.6.

### Example 82

### tert-butyl 3-[({4-[(Z)-cyano(1-ethyl-1,3-dihydro-2H-benzimidazol-2-ylidene)methyl]-5-methylpyrimidin-2-yl}amino)carbonyl]pyrrolidine-1-carboxylate

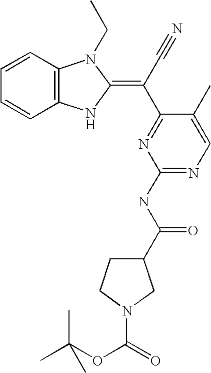

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and boc-1-pyrrolidine-3-carboxylic acid, the title compound was isolated, as a yellow solid in 70% yield (98% purity by HPLC).

MS(ESI⁺): 490.4; MS(ESI⁻): 488.3.

### Example 83

### N-{4-[(Z)-cyano(1-ethyl-1,3-dihydro-2H-benzimidazol-2-ylidene)methyl]-5-methylpyrimidin-2-yl}pyrrolidine-3-carboxamide

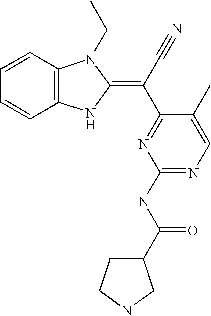

Following the general method as outlined in Example 57, starting tert-butyl 3-[({4-[(Z)-cyano(1-ethyl-1,3-dihydro-2H-benzimidazol-2-ylidene)methyl]-5-methylpyrimidin-2-yl}amino)carbonyl]pyrrolidine-1-carboxylate (Example 82), the title compound was isolated, as a yellow solid in 96% yield (99% purity by HPLC).

MS(ESI⁺): 388.4; MS(ESI⁻): 386.4.

### Example 84

### tert-butyl 2-[({4-[(Z)-cyano(1-ethyl-1,3-dihydro-2H-benzimidazol-2-ylidene)methyl]-5-methylpyrimidin-2-yl}amino)carbonyl]morpholine-4-carboxylate

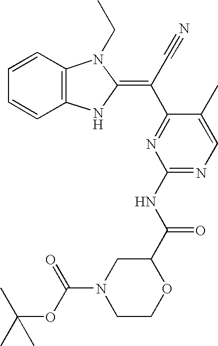

Following the general method as outlined in Example 56, starting from (2z)-(2-amino-5-methylpyrimidin)-b 4-yl)(1-ethyl-1,3-dihydro-2H-benzimidazol-2-ylidene)acetonitrile (Intermediate 19), and Boc-COP-OH compound was isolated, as a yellow solid in 70% yield (97% purity by HPLC).

MS(ESI⁺): 506.3 (ESI⁻): 504.6.

### Example 85

### N-{4-[(Z)-cyano(1-ethyl-1,3-dihydro-2H-benzimidazol-2-ylidene)methyl]-5-methylpyrimidin-2-yl}morpholine-2-carboxamide

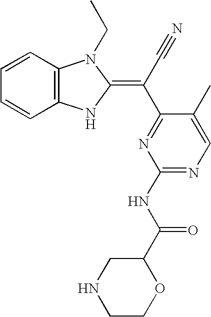

Following the general method as outlined in Example 56, starting from tert-butyl 2-[({4-[(Z)-cyano(1-ethyl-1,3-dihydro-2H-benzimidazol-2-ylidene)methyl]-5-methylpyrimidin-2-yl}amino)carbonyl]morpholine-4-carboxylate (Example 84), the title compound was isolated, as a yellow solid in 95% yield (99% purity by HPLC).

MS(ESI⁺): 406.6; MS(ESI⁻): 404.7.

### Example 86

### tert-butyl[2-({4-[(Z)-cyano(1-ethyl-1,3-dihydro-2H-benzimidazol-2-ylidene)methyl]-5-methylpyrimidin-2-yl}amino)-2-oxoethyl]methylcarbamate

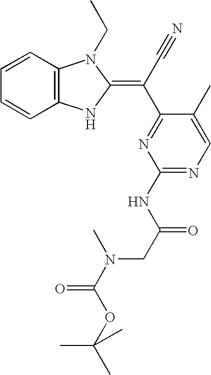

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and boc-sarcosine, the title compound was isolated, as a yellow solid in 82% yield (98% purity by HPLC).

MS(ESI⁺): 464.6; MS(ESI⁻): 462.2.

### Example 87

### N˜1˜˜{4-[(Z)-cyano(1-ethyl-1,3-dihydro-2H-benzimidazol-2-ylidene)-methyl]-5-methylpyrimidin-2-yl}-N˜2˜-methylglycinamide

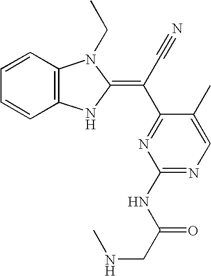

Following the general method as outlined in Example 56, starting from tert-butyl [2-({4-[(Z)-cyano(1-ethyl-1,3-dihydro-2H-benzimidazol-2-ylidene)methyl]-5-methylpyrimidin-2-yl}amino)-2-oxoethyl]methylcarbamate Example 86), the title compound was isolated, as a yellow solid in 98% yield (97% purity by HPLC).

MS(ESI⁺): 364.5; MS(ESI⁻): 362.5.

### Example 88

### N-{4-[(Z)-cyano(1-ethyl-1,3-dihydro-2H-benzimidazol-2-ylidene)methyl]-5-methylpyrimidin-2-yl}-1-methylpiperidine-3-carboxamide

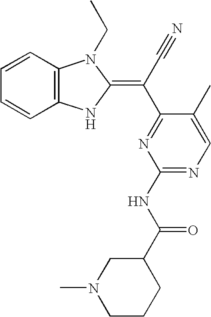

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 1-methyl-piperidine-3-carboxylic acid HCL, the title compound was isolated, as a yellow solid in 79% yield (94% purity by HPLC).

MS(ESI⁺): 418.9; MS(ESI⁻): 416.4.

### Example 89

### N-[3-({4-[(Z)-cyano(1-ethyl-1,3-dihydro-2H-benzimidazol-2-ylidene)-methyl]-5-methylpyrimidin-2-yl}amino)-3-oxopropyl]benzamide

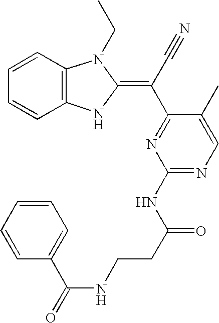

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and benzoyl-beta-alanine, the title compound was isolated, as a yellow solid in 40% yield (91% purity by HPLC).

MS(ESI⁺): 468.6; MS(ESI⁻): 466.1.

### Example 90

### tert-butyl 4-[2-({4-[(Z)-cyano(1ethyl-1,3-dihydro-2H-benzimidazol-2-ylidene)methyl]-5-methylpyrimidin-2-yl}amino)-2-oxoethyl]piperidine-1-carboxylate

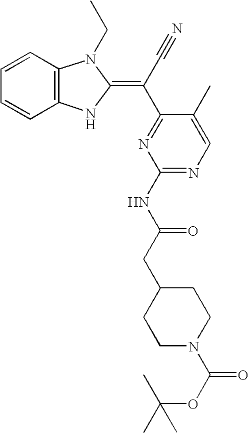

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 1-boc-4-piperidineacetic acid, the title compound was isolated, as a yellow solid in 70%/o yield (97% purity by HPLC).

MS(ESI⁺): 518.6; MS(ESI⁻): 516.2.

### Example 91

### 2-(1-acetylpiperidin-4-yl)-N-{4-[(Z)-cyano(1-ethyl-1,3-dihydro-2H-benzimidazol-2-ylidene)methyl]-5-methylpyrimidin-2-yl}acetamide

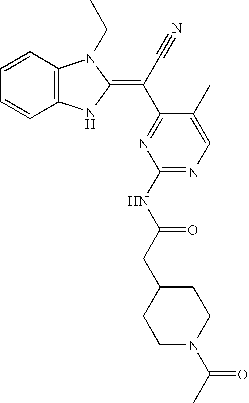

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 2-(N-acetylpiperidin-4yl)-carboxylic acid, the title compound was isolated, as a yellow solid in 85% yield (94% purity by HPLC).

MS(ESI⁺): 460.8; MS(ESI⁻): 458.3.

### Example 92

### N-{4-[(Z)-cyano(1-ethyl-1,3-dihydro-2H-benzimidazol-2-ylidene)methyl]-5-methylpyrimidin-2-yl}-2-piperidin-4-ylacetamide

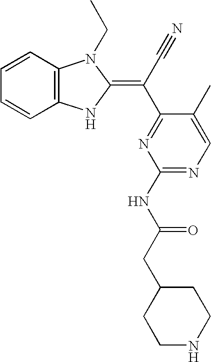

Following the general method as outlined in Example 57, starting from tert-butyl 4-[2-({4-[(Z)-cyano(1-ethyl-1,3-dihydro-2H-benzimidazol-2-ylidene)methyl]-5-methylpyrimidin-2-yl}amino)-2-oxoethyl]piperidine-1-carboxylate (Example 90), the title compound was isolated, as a yellow solid in 98% yield (97% purity by HPLC).

MS(ESI⁺): 418.6; MS(ESI⁻): 416.3.

### Example93

### tert-butyl 3-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate

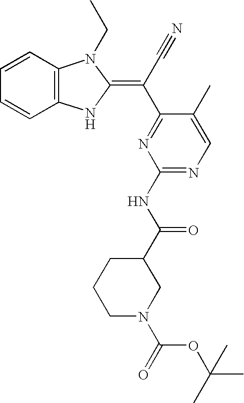

Following the general method as outlined in Example 56, starting from (2Z)-(2-amino-5-methylpyrimidin-4yl)(1-ethyl-1,3-dihydro-2H-benzimidazol-2-ylidene)acetonitrile (Intermediate 19), and (R,S)-boc-nipecotic acid, the title compound was isolated, as a yellow solid in 69% yield (99% purity by HPLC).

MS(ESI⁺): 504.6; MS(ESI⁻): 502.6.

### Example 94

### N-{4-[(Z)-cyano(1-ethyl-1,3-dihydro-2H-benzimidazol-2-ylidene)methyl]-5-methylpyrimidin-2-yl}piperidine-3-carboxamide

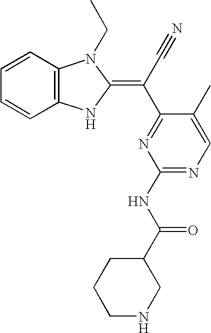

Following the general method as outlined in Example 57, starting from tert-butyl 3-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate (Example 93), the title compound was isolated, as a yellow solid in 95% yield (97% purity by HPLC).

MS(ESI⁺): 404.9; MS(ESI⁻): 402.6.

### Example 95

### N-{4-[(Z)-cyano(1-ethyl-1,3-dihydro-2H-benzimidazol-2-ylidene)methyl]-5-methylpyrimidin-2-yl}-4-[(4-methylpiperazin-1-yl)methyl]benzamide

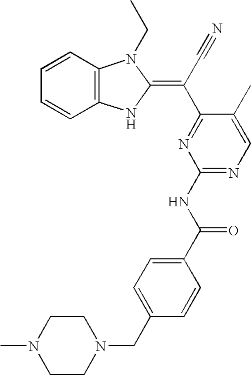

Following the general method as outlined in Example 56, starting from (2Z)-(2-amino-5-methylpyrimidin-4-yl)(1-ethyl-1,3 dihydro-2R-benzimidazol-2-ylidene)acetonitrile (Intermediate 19), and 4-[(4-methylpiperazin-1-yl)methyl]benzamide, the title compound was isolated, as a yellow solid in 58% yield (92% purity by HPLC).

MS(ESI⁺): 509.6; MS(ESI⁻): 507.5.

### Example 96

### 4-acetyl-N-{4-[(Z)-cyano-(1-ethyl-1,3-dihydro-2H-benzimidazol-2-ylidene)methyl]-5-methylpyrimidin-2-yl}morpholine-2-carboxamide

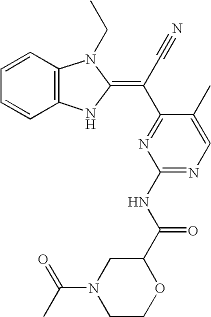

Following the general method as outlined in Example 56, starting from (2Z)(2-amino-5-methylpyrimidin-4yl)(1-ethyl-1,3-dihydro-2H-benzimidazol-2-ylidene)acetonitrile (Intermediate 19), and 4-acetyl-morpholine-2-carboxylic acid, the title compound was isolated, as a yellow solid in 62% yield (94% purity by HPLC).

MS(ESI⁺): 448.2; MS(ESI³¹ ): 446.3.

### Example 97

### N-{4-[(Z)-cyano(1-ethyl-1,3-dihydro-2H-benzimidazol-2-ylidene)methyl]-5-methylpyrimidin-2-yl}-4-methylmorpholine-2-carboxamide

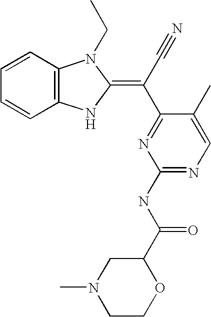

Following the general method as outlined in Example 56, staring from (2Z)-(2-amino-5-methylpyrimidin-4yl)(1-ethy-1,3dihydro-2H-benzimidazol-2-ylidene)acetonitrile (Intermediate 19), and 4-methylmorpholine-2-carboxylic acid, the title compound was isolated, as a yellow solid in 82% yield (96% purity by HPLC).

MS(ESI⁺): 420.6; MS(ESI⁻): 418.2.

### Example 98

### N-{4-[(Z)-cyano(1-ethyl-1,3-dihydro-2H-benzimidazol-2-ylidene)methyl]-5-methylpyrimidin-2-yl}-2-morpholin-4-ylacetamide

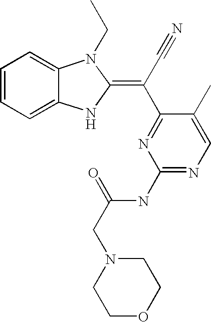

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 4-morpholinoacetic acid, the title compound was isolated, as a yellow solid in 82% yield (99% purity by HPLC).

MS(ESI): 420.1; MS(ESI⁻): 418.3.

### Example 99

### N˜2˜-benzyl-N˜1˜-{4-[(Z)-cyano(1-ethyl-1,3-dihydro-2 H-benzimidazol-2-ylidene)methyl]-5-methylpyrimidin-2-yl}glycinamide

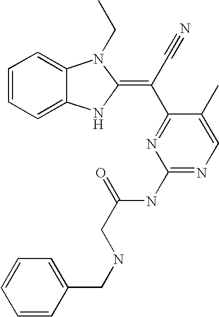

Following the general method as outlined in Example 56, starting from (2Z)(2-amino-5-methylpyrimidin-4-yl)(1-ethyl-1,3-dihydro-2H-benzimidazol-2-ylidene)acetonitrile (intermediate 19), and N-alpha-tert-butoxycarbonyl,benzyl-glycine which was further debocyclated by a solution of TFA:DCM (10:90), the title compound was isolated, as a yellow solid in 23% yield (97% purity by HPLC). MS(ESI⁺): 440.6; MS(ESI⁻): 438.5.

### Example 100

### N-{4-[(Z)-cyano(1-ethyl-1,3-dihydro-2H-benzimidazol-2-ylidene)methyl]-5-methylpyrimidin-2-yl}-2-(1,1-dioxidothiomoroholin-4-yl)acetamide

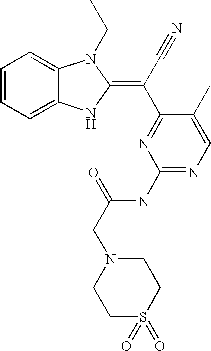

Following the genera method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and 4-thiomorpholine acetic acid, the title compound was isolated, as a yellow solid in 59% yield (98% purity by HPLC).

MS(ESI⁺): 468.6; MS(ESI⁻): 466.2.

### Example 101

### N˜1˜-{4-[(Z)-cyano(1-ethyl-1,3-dihydro-2-benzimidazol-2-ylidene)methyl]-5-methylpyrimidin-2-yl}-N˜2˜-formylglycinamide

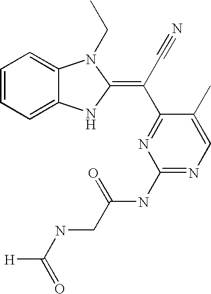

Following the general method as outlined in Example 56, starting from (2Z)-(2-amino-5-methylpyrimidin-4-yl)(1-ethyl-1,3-dihydro-2H-benzimidazol-2-ylidene)acetonitrile (Intermediate 19), and formyl glycine the title compound was isolated, as a yellow solid in 21% yield (96% purity by HPLC).

MS(ESI⁺): 378.8; MS(ESI⁻): 376.6.

### Example 102

### Preparation of a Pharmaceutical Formulation

The following formulation examples illustrate representative pharmaceutical compositions according to the present invention being not restricted thereto.

Formulation 1—Tablets

A benzimidazole acetonitrile of formula I is admixed as a dry powder with a dry gelatin binder in an approximate 1:2 weight ration. A minor amount of magnesium stearate is added as a lubricant. The mixture is formed into 240-270 mg tablets (80-90 mg of active benzimidazole acetonitrile compound per tablet) in a tablet press.

Formulation 2—Capsules

A benzimidazole acetonitrile of formula I is admixed as a dry powder with a starch diluent in an approximate 1:1 weight ratio. The mixture is filled into 250 mg capsules (125 mg of active benzimidazole acetonitrile compound per capsule).

Formulation 3—Liquid

A benzimidazole acetonile of formula I (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Tablets

A benzimidazole acetonitrile of formula I is admixed as a dry powder with a dry gelatin binder in an approximate 1:2 weight ratio. A minor amount of magnesium stearate is added as a lubricant. The mixture is formed into 450-900 mg tablets (150-300 mg of active benzimidazole acetonitrile compound) in a tablet press.

Formulation 5—Injection

A benzimidazole acetonitrile of formula (I) is dissolved in a buffered sterile saline injectable aqueous medium to a concentration of approximately 5 mg(ml.

Biological Assays

The compounds of the present invention may be subjected to the following assays:

a) GSK3 In Vitro Assay:

GSK3β Assay (see Bioorg. Med. Chem. Lett by Naerum et al. 12 p. 1525-1528 (2002))

In a final reaction volume of 25 μl, GSK3β (h) (5-10 mU) is incubated with 8 mM MOPS pH 7.0, 0.2 mM EDTA, 20 μM YRRAAVPPSPSLSRHSSPHQS(p)EDEEE (being the GSK3 substrate; a phospho GS2 peptide), 10 mM Mg Acetate and [γ-³³P-ATP] (Specific activity approx. 500 cpm/pmol, concentration as required). The reaction is initiated by the addition of Mg²⁺ [γ-³³P-ATP]. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and the degree of phosphorylation of the substrate is determined by scintillation counting.

The tested compounds according to formula I display an inhibition (IC₅₀) with regard to GSK3 of less than 20 μM, preferably less than 10 and even more preferred less than 1 μM.

The binding affinities of the compounds of formula (I) were assessed using the above described in vitro biological assay. Representative values for some example compounds are given in Tables 1 and 2 below.

The values in Table 1 refer to the binding affinity (IC₅₀; μM) of the example compounds according to formula I to GSK3.

TABLE 1
In vitro potency of benzimidazole derivatives on human GSK3 beta

| | | IC50 |
|---|---|---|
| | | (μM) |
| Structure | Compound | GSK3beta |
| 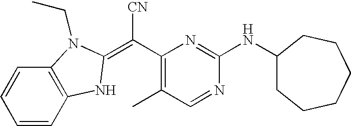 | (2Z)-[2-(cycloheptylamino)-5- methylpyrimidin-4-yl](1-ethyl-1,3- dihydro-2H-benzinaidazol-2- ylidene)acetonitrile | <10 |
| 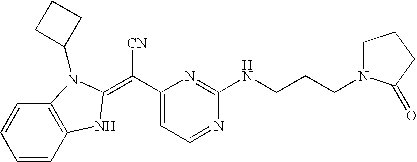 | (2Z)-(1-cyclobutyl-1,3-dihydro-2H- benzimidazol-2-ylidene)(2-{[3-(2- oxopyrrolidin-1- yl)propyl]amino}pyrinuidin-4- yl)acetonilrile | <10 |
| 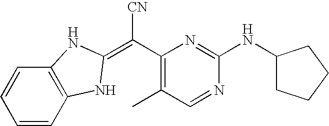 | [2-(cyclopentylamino)-5- methylpyrimidin-4-yl](1,3-dihydro- 2H-benzimidazol-2- ylidenc)acetonitrile | <10 |
| 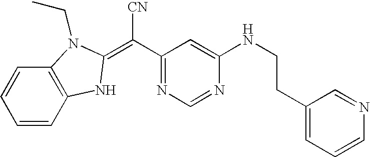 | (1-ethyl-1H-benzimidazol-2-yl){6-[(2- pyridin-3-ylethyl)amino]pyrimidin-4- yl}acetonitrile | <10 |
| 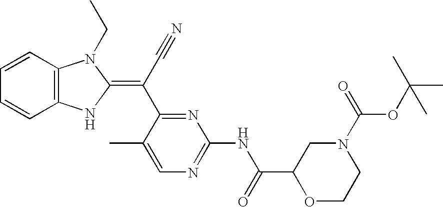 | tert-butyl 2-[({4-[(Z)-cyano(1-ethyl- 1,3-dihydro-2H-benzimidazol-2- ylidene)methyl]-5-methylpyrimidin-2- yl}amino)carbonyl]morpholine-4- carboxylate | <10 |
| 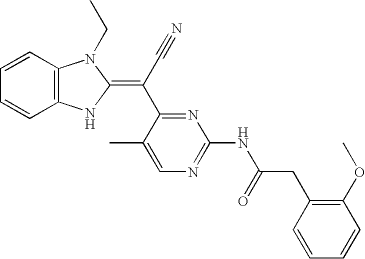 | N-{4-[(Z)-cyano(1-ethyl-1,3-dihydro- 2H-benzimidazol-2-ylidene)methyl]-5- methylpyrimidin-2-yl}-2-(2- methoxyphenyl)acetamide | <10 |
| 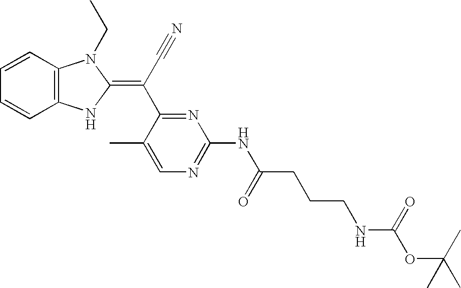 | tert-butyl [4-({4-[(Z)-cyano(1-ethyl- 1,3-dihydro-2H-benzimidazol-2- ylidene)methyl]-5-methylpyrimidin-2- yl}amino)-4-oxobutyl]carbamate | <10 |

b) In Vivo Assay: Experimental Model of Type II Diabetes (oral Postprandial Glycemia in db/db Mice)

The following assay aims at determining the anti-diabetic effect of the test compounds of formula (I) in a model of postprandial glycemia in db/db mice, in vivo.

The assay was performed as follows:

A total of 24 db/db mice (about 8-9 weeks; obtained from IFFACREDO, l'Arbreste, France) were fasted during 20 hours.

2 groups, each consisting of 6 animals were formed:
Group 1: The animals were administered (per os) a dose of 10 mg/kg of vehicle.
Group 2: The animals were administered per os) a dose of 50 mg/kg of the test compound according to formula (D.

After oral administration of the compounds of formula (I) solubilized or suspended in CarboxyMethylCellulose (0.5%), Tween 20 (0.25%) and water as vehicle, the animals had access to commercial food (D04, UAR, Villemoisson/Orge, France) ad libitum. The diabetic state of the mice was verified by determining the blood glucose level before drug administration. Blood glucose and serum insulin levels were then determined 4 hrs after drug administration.

The determination of the blood glucose level was performed using a glucometer (Precision Q.I.D., Medisense, Abbot, ref. 212.62.31).

The determination of the Insulin level was performed using an ELISA kit (Crystal CHEM, Ref. INSK R020).

Changes in blood glucose and serum insulin of drug treated mice were expressed as a percentage of control (group 1: vehicle treated mice).

Treatment (per os) of the animals with typical substituted benzimidazole acetonitrile compounds of formula (I), at a dosage of 50 mg/kg, decreased the blood glucose level induced by food intake by about 20-40%.

For instance, upon using the compound of example 3, i.e. (2Z)-[2-(cyclobutylamino)-5-methylpyrimidin-4-yl](1-ethyl-1,3-dihydro-2H-benzimidazol-2-ylidene)acetonitrile, the blood glucose level was found to be reduced at about 28% and the insulin level was found to be reduced at about 58%, compared to the animals of Group 1.

### REFERENCE LIST

1. Woodgett et al: Trends Biochem. Sci., 16 p. 177-81 (1991);
2. Reaven et al (American Journal of Medicine, 60, 80 (1976);
3. Stout, Metabolism, 34, 7 (1985)
4. Diamanti-Kandarakis et al.; European Journal of Endocrinology 138, 269-274 (1998),
5. Andrea Dunaif; Endocrine Reviews 18(6), 774-800 (1997));
6. WO 01/47920

1. A benzimidazole acetonitrile according to formula (I) 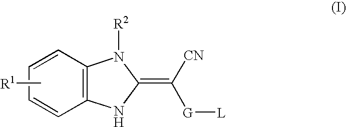
as well as its tautomers, its geometrical isomers, its optically active forms as enantiomers, diastereomers and its racemate forms, as well as pharmaceutically acceptable salts thereof, wherein
G is a pyrimidinyl;
L is an amino group, or a 3-8 membered heterocycloalkyl, containing at least one heteroatom selected from N, O, S or L is an acylamino moiety;
R¹ is selected from the group comprising or consisting of hydrogen, sulfonyl, amino, carboxy, amino carbonyl, C₁-C₆-alkyl, C₂-C₆-alkenyl, C₂-C₆-alkynyl or C₁-C₆-alkoxy, aryl, halogen, cyano or hydroxy;
R² is selected from the group comprising or consisting of hydrogen, C₁-C₆-alkyl, C₂-C₆-alkenyl, C₂-C₆-alkynyl, or C₁-C₆-alkoxy.

2. The benzimidazole acetonitrile according to claim 1, wherein R¹ is H or C₁-C₃ alkyl.

3. The benzimidazole acetonitrile according to claim 1, wherein R² is a C₁-C₃ alkyl.

4. The benzimidazole acetonitrile according to claim 1, having any of the formulae 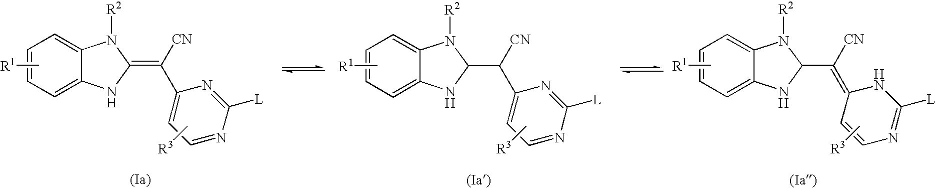
wherein R¹ is as above defined, and
R³ is selected from the group comprising or consisting of hydrogen, C₁-C₆-alkyl, C₂-C₆-alkenyl, C₂-C₆-alkynyl, or C₁-C₆-alkoxy;
L is an amino group of the formula —NR⁵R⁶ wherein R⁵ and R⁶ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, C₁-C₆-alkyl aryl, C₁-C₆-alkyl heteroaryl, C₁-C₆-alkenyl aryl, C₁-C₆-alkenyl heteroaryl, C₁-C₆-alkynyl aryl, C₁-C₆-alkynyl heteroaryl, C₁-C₆-alkyl cycloalkyl, C₁-C₆-alkyl heterocycloalkyl, C₁-C₆-alkenyl cycloalkyl, C₁-C₆-alkenyl heterocycloalkyl, C₁-C₆-alkynyl cycloalkyl, C₁-C₆-alkynyl heterocycloalkyl, or
R⁵ and R⁶ may form a ring together with the nitrogen to which they are bound.

5. The benzimidazole acetonitrile according to claim 1, wherein R⁵ is hydrogen or a methyl or ethyl or propyl group and R⁶ is a selected from the group consisting of H, (C₁-C₁₀)-alkyl, C₁-C₆ alkyl-aryl, C₁-C₆-alkyl-heteroaryl, cycloalkyl, heterocycloalkyl, aryl or heteroaryl and 4-8 membered saturated or unsaturated cycloalkyl.

6. The benzimidazole acetonitrile according to claim 1, wherein R⁵ is H and R⁶ is selected from the group consisting of C₁-C₆ alkyl, 3-8 membered cycloalkyl, 3-8 membered heterocycloalkyl, heteroaryl, C₁-C₆-alkyl heteroaryl, C₁-C₆-alkyl cycloalkyl, C₁-C₆-alkyl heterocycloalkyl.

7. The benzoxazole acetonitrile according to claim 1, wherein L is an acylamino moiety of the formula —NR⁵C(O)R⁶ wherein R⁵ and R⁶ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, C₁-C₆-alkyl aryl, C₁-C₆-alkyl heteroaryl, C₁-C₆-alkenyl aryl, C₁-C₆-alkenyl heteroaryl, C₁-C₆-alkynyl aryl, C₁-C₆-alkynyl heteroaryl, C₁-C₆-alkyl cycloalkyl, C, -C₆-alkyl heterocycloalkyl, C₁-C₆-alkenyl cycloalkyl, C₁-C₆-alkenyl heterocycloalkyl, C₁-C₆-alkynyl cycloalkyl, C₁-C₆-alkynyl heterocycloalkyl.

8. The benzimidazole acetonitrile according to claim 1 selected from the group consisting of:
(2Z)-(1-ethyl-1,3-dihydro-2H-benzimidazol-2-ylidene)(2-{[3-(1H-pyrazol-1-yl)propyl]amino}pyrimidin-4-yl)acetonitrile
(2Z)-(1-ethyl-1,3-dihydro-2H-benzimidazol-2-ylidene)(5-methyl-2-{[3-(1H-pyrazol-1-yl)propyl]amino}pyrimidin-4-yl)acetonitrile
(2Z)-[2-(cyclobutylamino)-5-methylpyrimidin-4-yl](1-ethyl-1,3-dihydro-2H-benzimidazol-2-ylidene)acetonitrile
(2Z)-(1-ethyl-1,3-dihydro-2H-benzimidazol-2-ylidene)(5-methyl-2-{[3-(2-oxopyrrolidin-1-yl)propyl]amino}pyrimidin-4-yl)acetonitrile
(2Z)-(1-ethyl-1,3-dihydro-2H-benzimidazol-2-ylidene)(2-{[3-(2-oxopyrrolidin-1-yl)propyl]amino}pyrimidin-4-yl)acetonitrile
(2Z)-(1-ethyl-1,3-dihydro-2H-benzimidazol-2-ylidene)(2-{[3-(1H-1,2,4-triazol-1-yl)propyl]amino}pyrimidin-4-yl)acetonitrile
(2Z)-(1-ethyl-1,3-dihydro-2H-benzimidazol-2-ylidene)(5-methyl-2-{[3-(1H-1,2,4-triazol-1-yl)propyl]amino}pyrimidin-4-yl)acetonitrile
[2-(cyclopentylamino)-5-methylpyrimidin-4-yl](1-ethyl-1H-benzimidazol-2-yl)acetonitrile
(2Z)-(2-{[3-(2-oxopyrrolidin-1-yl)propyl]amino}pyrimidin-4-yl)(1-propyl-1,3-dihydro-2H-benzimidazol-2-ylidene)acetonitrile
(1-ethyl-1H-benzimidazol-2-yl){5-methyl-2-[(2-pyridin-3-ylethyl)amino]pyrimidin-4-yl}acetonitrile
(2Z)-[2-(cyclobutylamino)pyrimidin-4-yl](1-ethyl-1,3-dihydro-2H-benzimidazol-2-ylidene)acetonitrile
(2Z)-[2-(cycloheptylamino)-5-methylpyrimidin-4-yl](1-ethyl-1,3-dihydro-2H-benzimidazol-2-ylidene)acetonitrile
[2-(cyclopentylamino)pyrimidin-4-yl](1-ethyl-1H-benzimidazol-2-yl)acetonitrile
1,3-dihydro-2H-benzimidazol-2-ylidene(5-methyl-2-{[3-(2-oxopyrrolidin-1-yl)propyl]amino}pyrimidin-4-yl)acetonitrile
(2Z)-(1-cyclobutyl-1,3-dihydro-2H-benzimidazol-2-ylidene)(2-{[3-(2-oxopyrrolidin-1-yl)propyl]amino}pyrimidin-4-yl)acetonitrile
(1-ethyl-1H-benzimidazol-2-yl){2-[(2-pyridin-3-yl-ethyl)amino]pyrimidin-4-yl}acetonitrile
(2Z)-(1-ethyl-1,3-dihydro-2H-benzimidazol-2-ylidene)[2-(isobutylamino)-5-methylpyrimidin-4-yl]acetonitrile
(2Z)-(1-ethyl-1,3-dihydro-2H-benzimidazol-2-ylidene)(2-{[2-(1H-imidazol-4-yl)ethyl]amino}pyrimidin-4-yl)acetonitrile
(2Z)-(1-ethyl-1,3-dihydro-2H-benzimidazol-2-ylidene)[2-(isobutylamino)pyrimidin-4-yl]acetonitrile
[2-(cyclopropylamino)pyrimidin-4-yl](1-ethyl-1H-benzimidazol-2-yl)acetonitrile
[2-({2-[6-(dimethylamino)pyridin-3-yl]ethyl}amino)pyrimidin-4-yl](1-ethyl-1H-benzimidazol-2-yl)acetonitrile
(1-ethyl-1H-benzimidazol-2-yl)(2-{[2-(1H-1,2,4-triazol-1-yl)ethyl]amino}pyrimidin-4-yl)acetonitrile
(2Z)-(1-ethyl-1,3-dihydro-2H-benzimidazol-2-ylidene)(2-{[2-(1H-imidazol-4-yl)ethyl]amino}-5-methylpyrimidin-4-yl)acetonitrile
[2-({2-[6-(dimethylamino)pyridin-3-yl]ethyl}amino)-5-methylpyrimidin-4-yl](1-ethyl-1H-benzimidazol-2-yl)acetonitrile
(2Z)-[2-(cycloheptylamino)pyrimidin-4-yl](1-ethyl-1,3-dihydro-2H-benzimidazol-2-ylidene)acetonitrile
[2-(cyclopropylamino)-5-methylpyrimidin-4-yl](1-ethyl-1H-benzimidazol-2-yl)acetonitrile
(1-ethyl-1H-benzimidazol-2-yl){2-[(2-pyridin-2-ylethyl)amino]pyrimidin-4-yl}acetonitrile
[2-(cyclopentylamino)-5-methylpyrimidin-4-yl](1,3-dihydro-2H-benzimidazol-2-ylidene)acetonitrile
[2-(cyclohexylamino)pyrimidin-4-yl](1-ethyl-1H-benzimidazol-2-yl)acetonitrile
(2Z)-(1-ethyl-1,3-dihydro-2H-benzimidazol-2-ylidene)(2-{[2-(1H-indol-3-yl)ethyl]amino}-5-methylpyrimidin-4-yl)acetonitrile
(1-ethyl-1H-benzimidazol-2-yl){5-methyl-2-[(2-pyridin-2-ylethyl)amino]pyrimidin-4-yl}acetonitrile
{2-[(2-ethoxyethyl)amino]pyrimidin-4-yl}(1-ethyl-1H-benzimidazol-2-yl)acetonitrile
(1-ethyl-1H-benzimidazol-2-yl){5-methyl-2-[(1-methylbutyl)amino]pyrimidin-4-yl}acetonitrile
(1-ethyl-1H-benzimidazol-2-yl)[2-(methylamino)pyrimidin-4-yl]acetonitrile
(1-ethyl-1H-benzimidazol-2-yl)(5-methyl-2-{[2-(1H-pyrazol-1-yl)ethyl]amino}pyrimidin-4-yl)acetonitrile
1H-benzimidazol-2-yl{5-methyl-2-[(2-pyridin-3-ylethyl)amino]pyrimidin-4-yl}acetonitrile
(2Z)-(1-ethyl-1,3-dihydro-2H-benzimidazol-2-ylidene)(2-{[2-(1H-imidazol-1-yl)ethyl]amino}-5-methylpyrimidin-4-yl)acetonitrile
1H-benzimidazol-2-yl{2-[(2-pyridin-3-ylethyl)amino]pyrimidin-4-yl}acetonitrile
(1-ethyl-1H-benzimidazol-2-yl){2-[(1-methylbutyl)amino]pyrimidin-4-yl}acetonitrile
{2-[(cyclohexylmethyl)amino]-5-methylpyrimidin-4-yl}(1-ethyl-1H-benzimidazol-2-yl)acetonitrile
1H-benzimidazol-2-yl[2-(cyclopentylamino)pyrimidin-4-yl]acetonitrile
(1-ethyl-1H-benzimidazol-2-yl){6-methyl-2-[(2-pyridin-3-ylethyl)amino]pyrimidin-4-yl}acetonitrile
1H-benzimidazol-2-yl[2-(cyclopropylamino)pyrimidin-4-yl]acetonitrile
[2-(cyclopentylamino)-6-methylpyrimidin-4-yl](1-ethyl-1H-benzimidazol-2-yl)acetonitrile
{2-[(cyclohexylmethyl)amino]pyrimidin-4-yl}(1-ethyl-1H-benzimidazol-2-yl)acetonitrile
(1-ethyl-1H-benzimidazol-2-yl){6-[(2-pyridin-3-ylethyl)amino]pyrimidin-4-yl}acetonitrile
(1-ethyl-1H-benzimidazol-2-yl){2-[(3-pyrrolidin-1-ylpropyl)amino]pyrimidin-4-yl}acetonitrile
(1-ethyl-1H-benzimidazol-2-yl)[2-(4-ethylpiperazin-1-yl)-5-methylpyrimidin-4-yl]acetonitrile
(1-ethyl-1H-benzimidazol-2-yl){2-[(2-furylmethyl)amino]-5-methylpyrimidin-4-yl}acetonitrile
(2Z)-(1-ethyl-1,3-dihydro-2H-benzimidazol-2-ylidene){5-methyl-2-[(1-methylpiperidin-4-yl)amino]pyrimidin-4-yl}acetonitrile
(2Z)-[2-(cyclohexylamino)-5-methylpyrimidin-4-yl](1-ethyl-1,3-dihydro-2H-benzimidazol-2-ylidene)acetonitrile
(2Z)-[2-(ethylamino)-5-methylpyrimidin-4-yl](1-ethyl-1,3-dihydro-2H-benzimidazol-2-ylidene)acetonitrile
[2-(cyclopentylamino)-5-methylpyrimidin-4-yl](1,3-diethyl-1,3-dihydro-2H-benzimidazol-2-ylidene)acetonitrile
(2Z)-(1-ethyl-1,3-dihydro-2H-benzimidazol-2-ylidene)[5-methyl-2-(piperidin-4-ylamino)pyrimidin-4-yl]acetonitrile
(2Z)-(1-ethyl-1,3-dihydro-2H-benzimidazol-2-ylidene){5-methyl-2-[(2-piperidin-1-ylethyl)amino]pyrimidin-4-yl}acetonitrile
N-{4-[(Z)-cyano(1-ethyl-1,3-dihydro-2H-benzimidazol-2-ylidene)methyl]-5-methylpyrimidin-2-yl}-4-(4-methylpiperazin-1-yl)-4-oxobutanamide
N-{4-[(Z)-cyano(1-ethyl-1,3-dihydro-2H-benzimidazol-2-ylidene)methyl]pyrimidin-2-yl}-4-(4-methylpiperazin-1-yl)-4-oxobutanamide
(1R,5R,7R)-N-{4-[(Z)-cyano(1-ethyl-1,3-dihydro-2H-benzimidazol-2-ylidene)methyl]-5-methylpyrimidin-2-yl}-6,8-dioxa-3-azabicyclo[3.2.1 ]octane-7-carboxamide
(1S,5S,7S)-N-{4-[(Z)-cyano(1-ethyl-1,3-dihydro-2H-benzimidazol-2-ylidene)methyl]-5-methylpyrimidin-2-yl}-6,8-dioxa-3-azabicyclo[3.2.1]octane-7-carboxamide
(1S,4S,5S,7R)-N-{4-[(Z)-cyano(1-ethyl-1,3-dihydro-2H-benzimidazol-2-ylidene)methyl]-5-methylpyrimidin-2-yl}-4-methyl-6,8-dioxa-3-azabicyclo[3.2.1]octane-7-carboxamide
(1S ,4R,5 S,7R)-N-{4-[(Z)-cyano(1-ethyl-1,3-dihydro-2H-benzimidazol-2-ylidene)methyl]-5-methylpyrimidin-2-yl}-4-methyl-6,8-dioxa-3-azabicyclo[3.2.1 ]octane-7-carboxamide
N-{4-[(Z)-cyano(1-ethyl-1,3-dihydro-2H-benzimidazol-2-ylidene)methyl]-5-methylpyrimidin-2-yl}piperazine-2-carboxamide
(4S)-N-{4-[(Z)-cyano(1-ethyl-1,3-dihydro-2H-benzimidazol-2-ylidene)methyl]-5-methylpyrimidin-2-yl}-1,3-thiazolidine-4-carboxamide
(4R)-N-{4-[(Z)-cyano(1-ethyl-1,3-dihydro-2H-benzimidazol-2-ylidene)methyl]-5-methylpyrimidin-2-yl}-1,3-thiazolidine-4-carboxamide
N-{4-[(Z)-cyano(1-ethyl-1,3-dihydro-2H-benzimidazol-2-ylidene)methyl]-5-methylpyrimidin-2-yl)-5-oxo-L-prolinamide
4-tert butyl 1-(9H-fluoren-9-ylmethyl)2-[({4-[(Z)-cyano(1-ethyl-1,3-dihydro-2H-benzimidazol-2-ylidene)methyl]-5-methylpyrimidin-2-yl}amino)carbonyl]piperazine-1,4-dicarboxylate
tert-butyl (4S)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate
tert-butyl (2S)-2-[({4-[(Z)-cyano(1-ethyl-1,3-dihydro-2H-benzimidazol-2-ylidene)methyl]-5-methylpyrimidin-2-yl }amino)carbonyl]-5-oxopyrrolidine-1-carboxylate
tert-butyl (4R)-4-[({4-[(Z)-cyano(1-ethyl-1,3-dihydro-2H-benzimidazol-2-ylidene)methyl]-5-methylpyrimidin-2-yl}amino)carbonyl]-1,3-thiazolidine-3-carboxylate
2-(1-acetylpiperidin-4-yl)-N-{4-[(Z)-cyano(1-ethyl-1,3-dihydro-2H-benzimidazol-2-ylidene)methyl]-5-methylpyrimidin-2-yl}acetamide
N-{4-[(Z)-cyano(1-ethyl-1,3-dihydro-2H-benzimidazol-2-ylidene)methyl]-5-methylpyrimidin-2-yl}-4-methylmorpholine-2-carboxamide
4-acetyl-N-{4-[(Z)-cyano(1-ethyl-1,3-dihydro-2H-benzimidazol-2-ylidene)methyl]-5-methylpyrimidin-2-yl}morpholine-2-carboxamide
N-{4-[(Z)-cyano(1-ethyl-1,3-dihydro-2H-benzimidazol-2-ylidene)methyl]-5-methylpyrimidin-2-yl}-4-[(4-methylpiperazin-1-yl)methyl]benzamide
tert-butyl 3-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate
tert butyl 4-[2-({4-[(Z)-cyano(1-ethyl-1,3-dihydro-2H-benzimidazol-2-ylidene)methyl]-5-methylpyrimidin-2-yl}amino)-2-oxoethyl]piperidine-1-carboxylate
N-{4-[(Z)-cyano(1-ethyl-1,3-dihydro-2H-benzimidazol-2-ylidene)methyl]-5-methylpyrimidin-2-yl}morpholine-2-carboxamide
N-{4-[(Z)-cyano(1-ethyl-1,3-dihydro-2H-benzimidazol-2-ylidene)methyl]-5-methylpyrimidin-2-yl}-2-piperidin-4-ylacetamide
N-{4-[(Z)-cyano(1-ethyl-1,3-dihydro-2H-benzimidazol-2-ylidene)methyl]-5-methylpyrimidin-2-yl}piperidine-3-carboxamide
tert-butyl 2-[({4-[(Z)-cyano(1-ethyl-1,3-dihydro-2H-benzimidazol-2-ylidene)methyl]-5-methylpyrimidin-2-yl}amino)carbonyl]morpholine-4-carboxylate
N-[3-({4-[(Z)-cyano(1-ethyl-1,3-dihydro-2H-benzimidazol-2-ylidene)methyl]-5-methylpyrimidin-2-yl}amino)-3-oxopropyl]benzamide
tert-butyl [2-({4-[(Z)-cyano(1-ethyl-1,3-dihydro-2H-benzimidazol-2-ylidene)methyl]-5-methylpyrimidin-2-yl}amino)-2-oxoethyl]methylcarbamate
N-(4-[(Z)-cyano(1-ethyl-1,3-dihydro-2H-benzimidazol-2-ylidene)methyl]-5-methylpyrimidin-2-yl}-1-methylpiperidine-3-carboxamide
N˜1˜-{4-[(Z)-cyano(1-ethyl-1,3-dihydro-2H-benzimidazol-2-ylidene)methyl]-5-methylpyrimidin-2-yl}-N˜2˜-methylglycinamide
N-{4-[(Z)-cyano(1-ethyl-1,3-dihydro-2H-benzimidazol-2-ylidene)methyl]-5-methylpyrimidin-2-yl}pyrrolidine-3-carboxamide
tert-butyl 3-[({4-[(Z)-cyano(1-ethyl-1,3-dihydro-2H-benzimidazol-2-ylidene)methyl]-5-methylpyrimidin-2-yl}amino)carbonyl]pyrrolidine-1-carboxylate
(2S)-N-(4-[(Z)-cyano(1-ethyl-1,3-dihydro-2H-benzimidazol-2-ylidene)methyl]-5-methylpyrimidin-2-yl)-2,5-dihydro-1H-pyrrole-2-carboxamide
tert-butyl (2S)-2-[({4-[(Z)-cyano(1-ethyl-1,3-dihydro-2H-benzimidazol-2-ylidene)methyl]-5-methylpyrimidin-2-yl}amino)carbonyl]-2,5-dihydro-1H-pyrrole-1-carboxylate
N-{4-[(Z)-cyano(1-ethyl-1,3-dihydro-2H-benzimidazol-2-ylidene)methyl]-5-methylpyrimidin-2-yl}-2-(2-methoxyphenyl)acetamide
1-acetyl-N-{4-[(Z)-cyano(1-ethyl-1,3-dihydro-2H-benzimidazol-2-ylidene)methyl]-5-methylpyrimidin-2-yl}piperidine-4-carboxamide
N-{4-[(Z)-cyano(1-ethyl-1,3-dihydro-2H-benzimidazol-2-ylidene)methyl]-5-methylpyrimidin-2-yl}-1-methylpiperidine-4-carboxamide
4-amino-N-{4-[(Z)-cyano(1-ethyl-1,3-dihydro-2H-benzimidazol-2-ylidene)methyl]-5-methylpyrimidin-2-yl}butanamide
tert-butyl 4-[({4-[(Z)-cyano(1-ethyl-1,3-dihydro-2H-benzimidazol-2-ylidene)methyl]-5-methylpyrimidin-2-yl}amino)carbonyl]piperidine-1-carboxylate
N-{4-[(Z)-cyano(1-ethyl-1,3-dihydro-2H-benzimidazol-2-ylidene)methyl]-5-methylpyrimidin-2-yl}piperidine-4-carboxamide
tert-butyl [4-({4-[(Z)-cyano(1-ethyl-1,3-dihydro-2H-benzimidazol-2-ylidene)methyl]-5-methylpyrimidin-2-yl}amino)-4-oxobutyl]carbamate
N-{4-[(Z)-cyano(1-ethyl-1,3-dihydro-2H-benzimidazol-2-ylidene)methyl]-5-methylpyrimidin-2-yl}cyclopentanecarboxamide
N-{4-[(Z)-cyano(1-ethyl-1,3-dihydro-2H-benzimidazol-2-ylidene)methyl]-5-methylpyrimidin-2-yl)-4-(4-methylpiperazin-1-yl)butanamide
N˜1˜-{4-[(Z)-cyano(1-ethyl-1,3-dihydro-2H-benzimidazol-2-ylidene)methyl]-5-methylpyrimidin-2-yl}-N˜3˜,N˜3˜-dimethyl-beta-alaninamide
N-{4-[(Z)-cyano(1-ethyl-1,3-dihydro-2H-benzimidazol-2-ylidene)methyl]-5-methylpyrimidin-2-yl}-4-(dimethylamino)butanamide
(2Z)-(2-amino-5-methylpyrimidin-4-yl)(1-ethyl-1,3-dihydro-2H-benzimidazol-2-ylidene)acetonitrile
N-{4-[(Z)-cyano(1-ethyl-1,3-dihydro-2H-benzimidazol-2-yliene)methyl]-5-methylpyrimidin-2-yl}-2-morpholin-4-ylacetamide
N˜2˜-benzyl-N˜1˜-{4-[(Z)-cyano(1-ethyl-1,3-dihydro-2H-benzimidazol-2-ylidene)methyl]-5-methylpyrimidin-2-yl}glycinamide
N-{4-[(Z)-cyano(1-ethyl-1,3-dihydro-2H-benzimidazol-2-ylidene)methyl]-5-methylpyrimidin-2-yl}-2-(1,1-dioxidothiomorpholin-4-yl)acetamide
N˜1˜-{4-[(Z)-cyano(1-ethyl-1,3-dihydro-2H-benzimidazol-2-ylidene)methyl]-5-methylpyrimidin-2-yl}-N˜2˜-formylglycinamide.

9. A benzimidazole acetonitrile according to claim 1 for the preparation of a medicament.

10. A benzimidazole acetonitrile according to claim 1 in the preparation of a medicament for the prevention and/or treatment of metabolic disorders mediated by insulin resistance or hyperglycemia, comprising diabetes type II, inadequate glucose tolerance, insulin resistance, obesity, polycystic ovary syndrome (PCOS).

11. The benzimidazole acetonitrile according to claim 9 wherein the disease is diabetes type II.

12. A pharmaceutical composition containing a benzimidazole acetonitrile according to claim 1 and a pharmaceutically acceptable carrier, diluent or excipient thereof.

13. The composition according to claim 12, further comprising at least one supplementary drug selected from the group consisting of insulin, aldose reductase inhibitors, alpha-glucosidase inhibitors, sulfonyl urea agents, biguanides, thiazolidines, PPARs agonists, GSK-3 inhibitors.

14. The composition according to claim 13 wherein said supplementary drug is selected from the group consisting of a rapid acting insulin, an intermediate acting insulin, a long acting insulin, a combination of intermediate and rapid acting insulins, Minalrestat, Tolrestat, Sorbinil, Methosorbinil, Zopolrestat, Epalrestat, Zenarestat, Imirestat, Ponalrestat, ONO-2235, GP-1447, CT-112, BAL-ARI 8, AD-5467, ZD5522, M-16209, NZ-314, M-79175, SPR-210, ADN 138, or SNK-860, Miglitol, Acarbose, Glipizide, Glyburide, Chlorpropamide, Tolbutamide, Tolazamide, or Glimepriride.

15. A method of preparing a benzimidazole acetonitrile of formula (I) according to claim 1, comprising the following step: 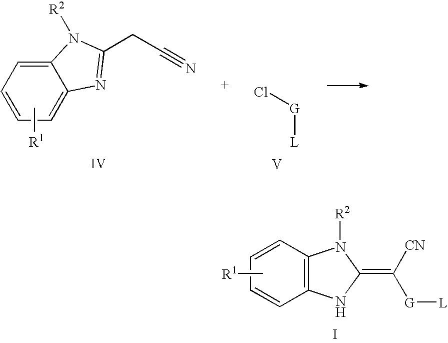
wherein R¹, R²,G, L are as above described.

16. A method of preparing a benzimidazole acetonitrile of formula (I) according to claim 1, comprising the following step:

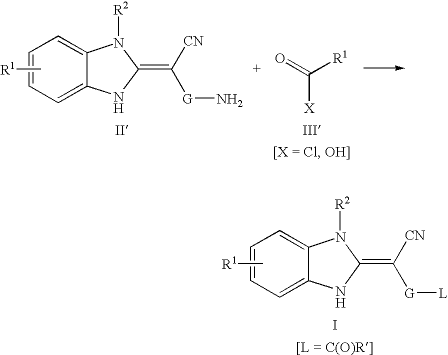

17. A method according to claim 15, comprising the following steps: 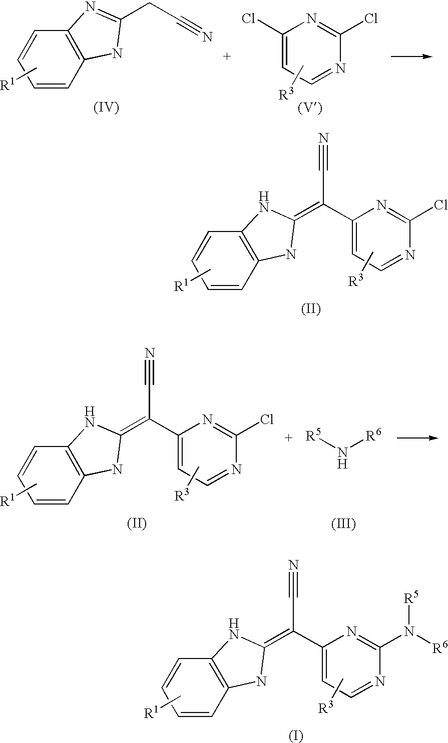
wherein R¹, R³ and R⁴ are as above defined.

17. (canceled)

18. (canceled)

19. The method according to claim 15, comprising the following steps: 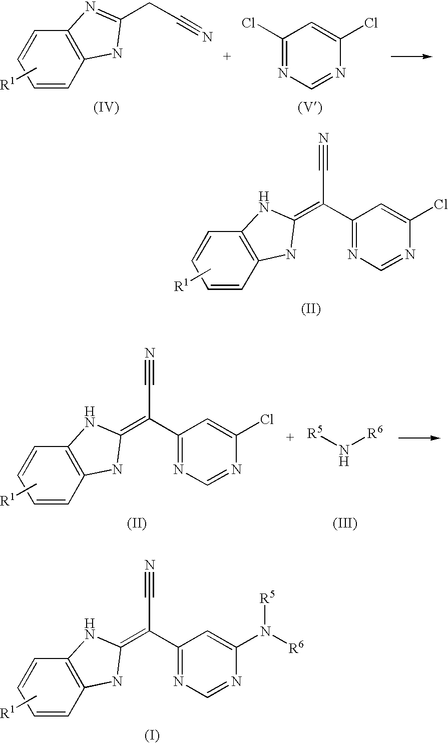
wherein R¹, R³ and R⁴ are as above defined.

20. The method according to claim 19, wherein the intermediate compound of formula (II) is selected from the group consisting of:
1,3-benzimidazol-2(3H)-ylidene(2-chloro-6-methylpyrimidin-4-yl)acetonitnle
1,3-benzimidazol-2(3H)-ylidene(2-chloro-6-methylpyrimidin-4-yl)acetonitrile
1,3-benzimidazol-2(3H)-ylidene(6-chloropyrimidin-4-yl)acetonitrile.
